SEQUENCE LISTING (255 sequences)

SEQ ID NO: 1 - DNA, 52 bp, unidentified
  source 1..52: mol_type = genomic DNA
gttttcacttcttgttccagacagccgcctgtttttccggatcatattgttc

SEQ ID NO: 2 - DNA, 52 bp, unidentified
  source 1..52: mol_type = genomic DNA
tgaccccgagggagccgcaccccggcgccgcgacgttcacctccgggggcgc

SEQ ID NO: 3 - DNA, 52 bp, unidentified
  source 1..52: mol_type = genomic DNA
gcgtccaccaggagctgggtgcctccgacctggcccaggacaagtatgtggc

SEQ ID NO: 4 - DNA, 52 bp, unidentified
  source 1..52: mol_type = genomic DNA
ctgaagtcactccaggctagaggggcggggcaatccccctcccctgcaatcc

SEQ ID NO: 5 - DNA, 52 bp, unidentified
  source 1..52: mol_type = genomic DNA
gattggtcgggaggccctcgagagtcggccaggcttcggggtgcctgtgggc

SEQ ID NO: 6 - DNA, 52 bp, unidentified
  source 1..52: mol_type = genomic DNA
ctggcccggaaggcgcagggcatctcgatgtcgtctccctcccggactgtca

SEQ ID NO: 7 - DNA, 52 bp, unidentified
  source 1..52: mol_type = genomic DNA
aagccacaggattggcacaaggctgcggtgaggtggggtggggatgctcagg

SEQ ID NO: 8 - DNA, 52 bp, unidentified
  source 1..52: mol_type = genomic DNA
ctgccgagggccggggaaggcctcccggactcctcgaaagcatctcgcaccg

SEQ ID NO: 9 - DNA, 52 bp, unidentified
  source 1..52: mol_type = genomic DNA
ccctccggcgcggcaccgtccctggcgggtctcgcacagctggaccctcgga

SEQ ID NO: 10 - DNA, 52 bp, unidentified
  source 1..52: mol_type = genomic DNA
ggccaaggaccgcacgtcccaggaccgggtggaggtgacggggcgcagcagg

SEQ ID NO: 11 - DNA, 52 bp, unidentified
  source 1..52: mol_type = genomic DNA
acttgtgttttttgtcaacatccttcgatgatatgcacttccttagaatcac

SEQ ID NO: 12 - DNA, 52 bp, unidentified
  source 1..52: mol_type = genomic DNA
ttcacagaccgcacactgaggctcgcggggctgcgacctactcaggccttcc

SEQ ID NO: 13 - DNA, 52 bp, unidentified
  source 1..52: mol_type = genomic DNA
aacctgatgaatgaggccagacggccggaagaacggcagaaataaatgcagg

SEQ ID NO: 14 - DNA, 52 bp, unidentified
  source 1..52: mol_type = genomic DNA
ggccgcgccaggacgatggagttgccggagagagaggggagcggcgcctctc

SEQ ID NO: 15 - DNA, 52 bp, unidentified
  source 1..52: mol_type = genomic DNA
cagcaccgggcatctggacagcaaccggagagcgaggcgtgtttctcctggt

SEQ ID NO: 16 - DNA, 52 bp, unidentified
  source 1..52: mol_type = genomic DNA
ctggacacggtgggagcgtcacagacgccaccagacactgcggagaggacgg

SEQ ID NO: 17 - DNA, 52 bp, unidentified
  source 1..52: mol_type = genomic DNA
ccggggcgcgtgcggaaccagcctccgcagagccctccaggccctacacgcg

SEQ ID NO: 18 - DNA, 52 bp, unidentified
  source 1..52: mol_type = genomic DNA
agtgggaggcctggagactgcgcagcgcttggttctcagggggccatgccct

SEQ ID NO: 19 - DNA, 52 bp, unidentified
  source 1..52: mol_type = genomic DNA
tcctctcccctctccccagcccccccgccgaggactaaggggcaagctagaa

SEQ ID NO: 20 - DNA, 52 bp, unidentified
  source 1..52: mol_type = genomic DNA
agcaaagcaggcacaattccaagagcgagacgatcccattcagcgcttctgg

SEQ ID NO: 21 - DNA, 52 bp, unidentified
  source 1..52: mol_type = genomic DNA
ccaagagcgagacgatcccattcagcgcttctgggtactgtgtcttgaggat

SEQ ID NO: 22 - DNA, 52 bp, unidentified
  source 1..52: mol_type = genomic DNA
cactcataataaaaacattatacgtcgtgtttgccggaggatgtctaaaata

SEQ ID NO: 23 - DNA, 52 bp, unidentified
  source 1..52: mol_type = genomic DNA
catcttgttgaccagactgacccctcggagcctaaacccccgagcggggccc

SEQ ID NO: 24 - DNA, 52 bp, unidentified
  source 1..52: mol_type = genomic DNA
ctacgagggtcttttacccattccacgcctgagaagaggccatgacgatcca

SEQ ID NO: 25 - DNA, 52 bp, unidentified
  source 1..52: mol_type = genomic DNA
gcggcgggggtgctgcgcggcgtcccgtgcggttgcaggctgggcgggaggc

SEQ ID NO: 26 - DNA, 52 bp, unidentified
  source 1..52: mol_type = genomic DNA
attcaccacaaaaaggaatgaagtacggacatacacaaggacgcaggtaaac

SEQ ID NO: 27 - DNA, 52 bp, unidentified
  source 1..52: mol_type = genomic DNA
tgtaagagggcttcggagtggtcctcggccaagcgctaccagcaccgcaaaa

SEQ ID NO: 28 - DNA, 52 bp, unidentified
  source 1..52: mol_type = genomic DNA
actgttccagggtgtacaaatcccacgccaacatccaaaataatggcaaata

SEQ ID NO: 29 - DNA, 52 bp, unidentified
  source 1..52: mol_type = genomic DNA
gtggattcaggattatacccctacccgaccccagactgtgagctgcgggaga

SEQ ID NO: 30 - DNA, 52 bp, unidentified
  source 1..52: mol_type = genomic DNA
gcccccaccttcttgtttgcccaagcgcacccccatccttgtccctcttcca

SEQ ID NO: 31 - DNA, 52 bp, unidentified
  source 1..52: mol_type = genomic DNA
cacgttcattgacaaacgcgcggggcgaaggcctagtggagcgcgggtgggg

SEQ ID NO: 32 - DNA, 52 bp, unidentified
  source 1..52: mol_type = genomic DNA
gtccggcagcagcgcgcaccgggcccggccccgcgctccgggacgcgagcgg

SEQ ID NO: 33 - DNA, 52 bp, unidentified
  source 1..52: mol_type = genomic DNA
cacacggggcggggggcgcggtccccggggctgccagcgccgcggtcactca

SEQ ID NO: 34 - DNA, 52 bp, unidentified
  source 1..52: mol_type = genomic DNA
ccccgccccgctcacgctcaccgcgcggcgcaccactcaccggctctctccc

SEQ ID NO: 35 - DNA, 52 bp, unidentified
  source 1..52: mol_type = genomic DNA
gtcccccctgcaggctccaggctcccggacacactcggggttcaagtcaaga

SEQ ID NO: 36 - DNA, 52 bp, unidentified
  source 1..52: mol_type = genomic DNA
ccccaaaatatctcctctctgggtccggaaatccctcccaaccctctccgag

SEQ ID NO: 37 - DNA, 52 bp, unidentified
  source 1..52: mol_type = genomic DNA
ctcagccgcttgggcgggtatccaccggccccgggggtagtcggcccctgcg

SEQ ID NO: 38 - DNA, 52 bp, unidentified
  source 1..52: mol_type = genomic DNA
gtgagccagcctggtcaggggcggccggagccaaggacccgtggctccctca

SEQ ID NO: 39 - DNA, 52 bp, unidentified
  source 1..52: mol_type = genomic DNA
cctcctaaacgtgggccctgtgttccggagcccctggaatcgtttcagcatg

SEQ ID NO: 40 - DNA, 52 bp, unidentified
  source 1..52: mol_type = genomic DNA
cgcacctgacccccgttgcccactgcgaaatttctccaaagcttccatccca

SEQ ID NO: 41 - DNA, 52 bp, unidentified
  source 1..52: mol_type = genomic DNA
cggggcactccctggacaacagggacgcgctggccccaacgttaggcgcaat

SEQ ID NO: 42 - DNA, 52 bp, unidentified
  source 1..52: mol_type = genomic DNA
ccgacacctgcaacaacggcacctgcgggggcggaggggtgtcggctgggcg

SEQ ID NO: 43 - DNA, 52 bp, unidentified
  source 1..52: mol_type = genomic DNA
agtccagggaacagggccaaggtcacgcagaaaggtcactgttatcaaaacg

SEQ ID NO: 44 - DNA, 52 bp, unidentified
  source 1..52: mol_type = genomic DNA
ttcccggctcccgaaccagggtggccgagccaggcctctgcctccgggggtc

SEQ ID NO: 45 - DNA, 52 bp, unidentified
  source 1..52: mol_type = genomic DNA
ccctggtgggggccttaggcaaaaacgggtagccgcaagcagggacgcagcg

SEQ ID NO: 46 - DNA, 52 bp, unidentified
  source 1..52: mol_type = genomic DNA
tgcagccgggcctcctggggcagggcgagctcgcacctgctggcctccccgc

SEQ ID NO: 47 - DNA, 52 bp, unidentified
  source 1..52: mol_type = genomic DNA
tgacgctcccgcggcgcggctcccccgccgagcccacggcgctccggcccca

SEQ ID NO: 48 - DNA, 52 bp, unidentified
  source 1..52: mol_type = genomic DNA
ctcgcctgcgctcctcacctgggcgcgctgcgcggtcacggcagatgctcgg

SEQ ID NO: 49 - DNA, 52 bp, unidentified
  source 1..52: mol_type = genomic DNA
ggggcgacccctggactgtggctggcgtctgctgtgaggtggcctgtggggt

SEQ ID NO: 50 - DNA, 52 bp, unidentified
  source 1..52: mol_type = genomic DNA
tatctcagccattcatagcgcctaacggctcacaataccctaaaggctccct

SEQ ID NO: 51 - DNA, 52 bp, unidentified
  source 1..52: mol_type = genomic DNA
ccggcggtgccagcatgtccgccgccgtggcgtgcctggactacttcgccgc

SEQ ID NO: 52 - DNA, 52 bp, unidentified
  source 1..52: mol_type = genomic DNA
gagctttacctcccaccactccctccgcatgtcctgggctctggccattacc

SEQ ID NO: 53 - DNA, 52 bp, unidentified
  source 1..52: mol_type = genomic DNA
tggtagctggggcccccgcctgggccgccgtctccgccgcccgggccgctgt

SEQ ID NO: 54 - DNA, 52 bp, unidentified
  source 1..52: mol_type = genomic DNA
tttctaggactacgtctgtgcacctcgtgtggcaaataaaactatgctaaca

SEQ ID NO: 55 - DNA, 52 bp, unidentified
  source 1..52: mol_type = genomic DNA
cctctagaccccaacccttcgaggtcggtctcaggtccggtctcaggtctcg

SEQ ID NO: 56 - DNA, 52 bp, unidentified
  source 1..52: mol_type = genomic DNA
aataactcgtcaagtccctgggctccgaggcagcctgcctccccaccagccc

SEQ ID NO: 57 - DNA, 52 bp, unidentified
  source 1..52: mol_type = genomic DNA
ccccagccgggccggctcgggtttccgacggagggccgggctgcgcggcgtc

SEQ ID NO: 58 - DNA, 52 bp, unidentified
  source 1..52: mol_type = genomic DNA
ggcagccggggcagcgggaggcgtgcgcagcgccggcgaggaggacccgcgc

SEQ ID NO: 59 - DNA, 52 bp, unidentified
  source 1..52: mol_type = genomic DNA
caccgctgagcttcccgccgccctgcgcagcgcagccctctcgccgcttata

SEQ ID NO: 60 - DNA, 52 bp, unidentified
  source 1..52: mol_type = genomic DNA
agtttctttttgggaagatgaaccccgacggaggcagctgcgcggggtgtgt

SEQ ID NO: 61 - DNA, 52 bp, unidentified
  source 1..52: mol_type = genomic DNA
gagtttctgtgcacgaggtcatcctcggtcaagtattattcccacccgtctc

SEQ ID NO: 62 - DNA, 52 bp, unidentified
  source 1..52: mol_type = genomic DNA
ccggccggccggccacggaaacctccggaggcccgggccccgccggggtgtc

SEQ ID NO: 63 - DNA, 52 bp, unidentified
  source 1..52: mol_type = genomic DNA
ggcgggctccccgtgcgcgcacggccgcggccgacctctccgcgcgggctcc

SEQ ID NO: 64 - DNA, 52 bp, unidentified
  source 1..52: mol_type = genomic DNA
cataactgatgcccacagggtctgacgttctgagttaacctgcattataact

SEQ ID NO: 65 - DNA, 52 bp, unidentified
  source 1..52: mol_type = genomic DNA
acgcggcatagcgggctccgcgctccggcaggggtcggtcgtcgaaggtcgg

SEQ ID NO: 66 - DNA, 52 bp, unidentified
  source 1..52: mol_type = genomic DNA
tggatagagaggggtcccttaaaagcgtgccttgagttcaggctttgaaaaa

SEQ ID NO: 67 - DNA, 52 bp, unidentified
  source 1..52: mol_type = genomic DNA
ctgggggctacgagcgagaatccctcgacgctcctgagaaactcagaccaga

SEQ ID NO: 68 - DNA, 52 bp, unidentified
  source 1..52: mol_type = genomic DNA
acagcaggatgaagccgaaagcctccgagtgccggtcgaagaagtactcgtt

SEQ ID NO: 69 - DNA, 52 bp, unidentified
  source 1..52: mol_type = genomic DNA
tcctgggcccgcggcgctggcccctcgggcctcgctgaaagccccttgttcc

SEQ ID NO: 70 - DNA, 52 bp, unidentified
  source 1..52: mol_type = genomic DNA
atgctactgcaggcactggctgcttcggcgtggctctgtgcggtagtgtctc

SEQ ID NO: 71 - DNA, 52 bp, unidentified
  source 1..52: mol_type = genomic DNA
gaagcgaggagccgcctcccgaggccggtcccggcgagcaagggcgcctcgg

SEQ ID NO: 72 - DNA, 52 bp, unidentified
  source 1..52: mol_type = genomic DNA
cgggtgtcctgtgtgtatctgtgtgcgtgtttgtttaatcctcccccaagct

SEQ ID NO: 73 - DNA, 52 bp, unidentified
  source 1..52: mol_type = genomic DNA
acaatccttttaaggcaacgtgtcccgcctctcctaaccatataagtgcaac

SEQ ID NO: 74 - DNA, 52 bp, unidentified
  source 1..52: mol_type = genomic DNA
cagacgcgagcgaggccggtggaggcggggtggctgcgttggacgggacggg

SEQ ID NO: 75 - DNA, 52 bp, unidentified
  source 1..52: mol_type = genomic DNA
ggtgccgtcggcgaagcgggccagccggttggagctgcgcccgcagccgccg

SEQ ID NO: 76 - DNA, 52 bp, unidentified
  source 1..52: mol_type = genomic DNA
gtggcctgggcaggtcagcggagcccgcggcgctcagcgccccacctgtatg

SEQ ID NO: 77 - DNA, 52 bp, unidentified
  source 1..52: mol_type = genomic DNA
ggcgcgatgcgggcggccgcggcggcgtctacgagcacctcggcggggcgcc

SEQ ID NO: 78 - DNA, 52 bp, unidentified
  source 1..52: mol_type = genomic DNA
tcccggaagtcctgctcaactagcccggctggcggcccggtgtcacccgctg

SEQ ID NO: 79 - DNA, 52 bp, unidentified
  source 1..52: mol_type = genomic DNA
gtgccaccatccccgggttgaaggacgggttccgagtgagccaggaaccacc

SEQ ID NO: 80 - DNA, 52 bp, unidentified
  source 1..52: mol_type = genomic DNA
ggatccgtcattgacccttggagaacggaagattctgggggctcaaaggagt

SEQ ID NO: 81 - DNA, 52 bp, unidentified
  source 1..52: mol_type = genomic DNA
ccaacatgacgctcatcggagagaacggccacagcaccgacacgctcacggg

SEQ ID NO: 82 - DNA, 52 bp, unidentified
  source 1..52: mol_type = genomic DNA
ttaagcccacgctaatttccataggcgagttagaatgtggcctaaattgctt

SEQ ID NO: 83 - DNA, 52 bp, unidentified
  source 1..52: mol_type = genomic DNA
cccacagcccctcgcgtccccggagcgcgcacgcccgcagcggtgcaaactg

SEQ ID NO: 84 - DNA, 52 bp, unidentified
  source 1..52: mol_type = genomic DNA
agaaggatctggaacgtgtaggagacgtgcagcttctcagggtccgtctcca

SEQ ID NO: 85 - DNA, 52 bp, unidentified
  source 1..52: mol_type = genomic DNA
gcccatcgcctgcggcctcggtctccggggagagggaaagtaactccggagc

SEQ ID NO: 86 - DNA, 52 bp, unidentified
  source 1..52: mol_type = genomic DNA
ttggaagctcggggggcggagatcccgcgccgcccggccggcctcggggcgg

SEQ ID NO: 87 - DNA, 52 bp, unidentified
  source 1..52: mol_type = genomic DNA
ctgcacagccctgctccgccctgcgcggccctgcacggtgctgctcggtgct

SEQ ID NO: 88 - DNA, 52 bp, unidentified
  source 1..52: mol_type = genomic DNA
ttgagtgacggctagcggcccgccccgggcgtgacgtcatcccggtgttgct

SEQ ID NO: 89 - DNA, 52 bp, unidentified
  source 1..52: mol_type = genomic DNA
cttcacagtaagcctaaagagtaggcgttatctccgtttatggtgtaactgg

SEQ ID NO: 90 - DNA, 52 bp, unidentified
  source 1..52: mol_type = genomic DNA
cgccagcctgcagcaggtccatgagcggacgtgggcccggcagttgctgcag

SEQ ID NO: 91 - DNA, 52 bp, unidentified
  source 1..52: mol_type = genomic DNA
aaaagccaggggagaggggaggcgccggctcccacaggccactgtaagtccc

SEQ ID NO: 92 - DNA, 52 bp, unidentified
  source 1..52: mol_type = genomic DNA
agtaaggagctgctctcatgactgtcgttgctgttgctaagtggcccttttc

SEQ ID NO: 93 - DNA, 52 bp, unidentified
  source 1..52: mol_type = genomic DNA
ccgcggccccactcggtcacgcccacgggcaccctcacggagcctccaggga

SEQ ID NO: 94 - DNA, 52 bp, unidentified
  source 1..52: mol_type = genomic DNA
cccgcgcgctgccgcggccgcctgtcggccaaggagctcaaccacgtcctgc

SEQ ID NO: 95 - DNA, 52 bp, unidentified
  source 1..52: mol_type = genomic DNA
cttcattgcatcgccttcccgacgccgggtgccaacaagcaaccaaaaaatt

SEQ ID NO: 96 - DNA, 52 bp, unidentified
  source 1..52: mol_type = genomic DNA
ccaggattccacatggtcaggagaacgctctctgcacatcatggacaccaaa

SEQ ID NO: 97 - DNA, 52 bp, unidentified
  source 1..52: mol_type = genomic DNA
ttctcccgcctcagcacctccagcgcggacagctcctcgctgtcgcctccac

SEQ ID NO: 98 - DNA, 52 bp, unidentified
  source 1..52: mol_type = genomic DNA
ggcagggccgccaggcgccggcccccggtgaagaagtcctcggcgctgcgct

SEQ ID NO: 99 - DNA, 52 bp, unidentified
  source 1..52: mol_type = genomic DNA
ttgcgtaggaggtaggaggcgaggccgtggggctgggaacccgaactgggct

SEQ ID NO: 100 - DNA, 52 bp, unidentified
  source 1..52: mol_type = genomic DNA
caagtcacccagctcatctgttgagcgttcactgtcctaagccctttatgac

SEQ ID NO: 101 - DNA, 52 bp, unidentified
  source 1..52: mol_type = genomic DNA
tcggtgccgcccgcctcgggctctgcgcgcggccagcgctgcgggtgacaga

SEQ ID NO: 102 - DNA, 52 bp, unidentified
  source 1..52: mol_type = genomic DNA
tgaaattttcgagtctcggagacggcgtggatgctgggggtcctgaaagcct

SEQ ID NO: 103 - DNA, 52 bp, unidentified
  source 1..52: mol_type = genomic DNA
acctgcccatgggggataataataacgttaatgaaacccttgcagagtactc

SEQ ID NO: 104 - DNA, 52 bp, unidentified
  source 1..52: mol_type = genomic DNA
accacgccccgggggccgagcgtcgcgggggtcggacgccgagcagctgccg

SEQ ID NO: 105 - DNA, 52 bp, unidentified
  source 1..52: mol_type = genomic DNA
ccctcaggaactcctccaggtccagcgtcccgctgccatcacggtcccagcg

SEQ ID NO: 106 - DNA, 52 bp, unidentified
  source 1..52: mol_type = genomic DNA
accagccgcccagccgtgacctttccgggcctgttcctgcggctctgggcgc

SEQ ID NO: 107 - DNA, 52 bp, unidentified
  source 1..52: mol_type = genomic DNA
gcggcagctcgggaagtcaaccccccggcgacatctgacagggcaggccggc

SEQ ID NO: 108 - DNA, 52 bp, unidentified
  source 1..52: mol_type = genomic DNA
agcgcaggacacccctgcacgtcggcgccgcgggcggtgccaaggtcctggg

SEQ ID NO: 109 - DNA, 52 bp, unidentified
  source 1..52: mol_type = genomic DNA
cggcgggcgcgctgctggcgggcgccgagggcggggacgtgcgcgaggccac

SEQ ID NO: 110 - DNA, 52 bp, unidentified
  source 1..52: mol_type = genomic DNA
agaataagtccgtcttcatcctgggcgccagcggggaaaccggcagagtgct

SEQ ID NO: 111 - DNA, 52 bp, unidentified
  source 1..52: mol_type = genomic DNA
acgcgctggcccgcgctgcccgccgcgctcgggagagcatccctgccgctgc

SEQ ID NO: 112 - DNA, 52 bp, unidentified
  source 1..52: mol_type = genomic DNA
ttggacgcggaggccgtccgggcgccgagcaacgagcagcacagcacggtgg

SEQ ID NO: 113 - DNA, 52 bp, unidentified
  source 1..52: mol_type = genomic DNA
atccagagggggctcctggggcctgcgttgccggagcaccccaagcaaatac

SEQ ID NO: 114 - DNA, 52 bp, unidentified
  source 1..52: mol_type = genomic DNA
agggggaggtgacatgggaattaaacgccctttccctcagccagtgccaaat

SEQ ID NO: 115 - DNA, 52 bp, unidentified
  source 1..52: mol_type = genomic DNA
cggggcgggaatccgcccgcccgctcgcccccggcgcctagacccggggcgc

SEQ ID NO: 116 - DNA, 52 bp, unidentified
  source 1..52: mol_type = genomic DNA
cccagaaactacggcaaactcgcgccgggtgcgcgctggaagggggagtccg

SEQ ID NO: 117 - DNA, 52 bp, unidentified
  source 1..52: mol_type = genomic DNA
ctcttcctcggagatccatgctcgccgtgcagcgaaggaggccagatgtgct

SEQ ID NO: 118 - DNA, 52 bp, unidentified
  source 1..52: mol_type = genomic DNA
tgggtaggtgagagcagggcaggggcgccaagttcgggctgcccagactgct

SEQ ID NO: 119 - DNA, 52 bp, unidentified
  source 1..52: mol_type = genomic DNA
acgttgctcttcgtgctcctgccctcgctggtcgtgcagctgctcagcttcc

SEQ ID NO: 120 - DNA, 52 bp, unidentified
  source 1..52: mol_type = genomic DNA
cagaaccgcgcttccccgaaactcgcgggcggtgggatcgcaagtcaccccc

SEQ ID NO: 121 - DNA, 52 bp, unidentified
  source 1..52: mol_type = genomic DNA
gtcctctggcgagggaagttacagacgccaagcccagagtctggggtgccca

SEQ ID NO: 122 - DNA, 52 bp, unidentified
  source 1..52: mol_type = genomic DNA
cgccgggccccaggagccgccgtcacgaccgccgcctcgcgtagcgcctccg

SEQ ID NO: 123 - DNA, 52 bp, unidentified
  source 1..52: mol_type = genomic DNA
accaccgacaaaagataatgttgcccgaataagttacgtgcatcttaacatg

SEQ ID NO: 124 - DNA, 52 bp, unidentified
  source 1..52: mol_type = genomic DNA
aagttacgtgcatcttaacatgtcacgggatctgttcctgtgtgttcttgtg

SEQ ID NO: 125 - DNA, 52 bp, unidentified
  source 1..52: mol_type = genomic DNA
cgctcgagcatctactctactgcggcgaatttaatccacatttcctctgcat

SEQ ID NO: 126 - DNA, 52 bp, unidentified
  source 1..52: mol_type = genomic DNA
agagccaggcgcggccccgcgaggccgggtgcacgtgcacacgaggggcgcc

SEQ ID NO: 127 - DNA, 52 bp, unidentified
  source 1..52: mol_type = genomic DNA
cttgaggccttgaggatggcccctgcgtccacgaccagggctggggctcctg

SEQ ID NO: 128 - DNA, 52 bp, unidentified
  source 1..52: mol_type = genomic DNA
ccgggacccggaacgcaggcgccgccgaccctcagtcccgggccgcgtgcat

SEQ ID NO: 129 - DNA, 52 bp, unidentified
  source 1..52: mol_type = genomic DNA
tcccagcgtttcagctcctgagtgtcggctggaagcccgccagggttaccat

SEQ ID NO: 130 - DNA, 52 bp, unidentified
  source 1..52: mol_type = genomic DNA
cccggcagtactggctcttcgcccgcgagcacccgcaccacgcgccgctgcg

SEQ ID NO: 131 - DNA, 52 bp, unidentified
  source 1..52: mol_type = genomic DNA
tgtattaactccataaatgtatatacgggatagaaggacctgccctgtctcc

SEQ ID NO: 132 - DNA, 52 bp, unidentified
  source 1..52: mol_type = genomic DNA
taattccattctggtttgtagaattcgtctgcaggctattgtggataggata

SEQ ID NO: 133 - DNA, 52 bp, unidentified
  source 1..52: mol_type = genomic DNA
ccctgagcagtgccacccttcttcccggaaaaggagggaagccatccttttc

SEQ ID NO: 134 - DNA, 52 bp, unidentified
  source 1..52: mol_type = genomic DNA
caacatgcaactcggggttgttctgcggatgtgtttaaggccaatgtaattc

SEQ ID NO: 135 - DNA, 52 bp, unidentified
  source 1..52: mol_type = genomic DNA
ccagcggccgccagccctgcgccgccgcgacacaaagagtgcgggcgattcc

SEQ ID NO: 136 - DNA, 52 bp, unidentified
  source 1..52: mol_type = genomic DNA
gagcccgccaagttagaagactggtcggtcggggccacacggctggtccggg

SEQ ID NO: 137 - DNA, 52 bp, unidentified
  source 1..52: mol_type = genomic DNA
tagtagtcgatagaggactggaacacggcacttcagataaagaatgctccag

SEQ ID NO: 138 - DNA, 52 bp, unidentified
  source 1..52: mol_type = genomic DNA
actagattttgaattctatgatgaccgggactactactacttctacctttgc

SEQ ID NO: 139 - DNA, 52 bp, unidentified
  source 1..52: mol_type = genomic DNA
tggggaggtggggaatcgcgtaccacggtggcagcttgctgtaccagggcag

SEQ ID NO: 140 - DNA, 52 bp, unidentified
  source 1..52: mol_type = genomic DNA
gcaggtggtgggacagccattgggccgtgtagaaccgaggttcccacgtttg

SEQ ID NO: 141 - DNA, 52 bp, unidentified
  source 1..52: mol_type = genomic DNA
tcccgatgatggtgattgatcattacgaggacggtaataagattgttacccg

SEQ ID NO: 142 - DNA, 52 bp, unidentified
  source 1..52: mol_type = genomic DNA
cccgcccgcactcccggcccgcgcccggaggcagtgtggggtgcgggcctcc

SEQ ID NO: 143 - DNA, 52 bp, unidentified
  source 1..52: mol_type = genomic DNA
tggagcggtgcgcgatgctgtctcacgccgacctcctggacgccaggctagg

SEQ ID NO: 144 - DNA, 52 bp, unidentified
  source 1..52: mol_type = genomic DNA
tctcgcccgggcaaccaggagaagccggggcaggaaattgtcaatttgttcc

SEQ ID NO: 145 - DNA, 52 bp, unidentified
  source 1..52: mol_type = genomic DNA
gctgagcccgcagctgcggcgcggacgccggtcgcacgcccggcggggctgg

SEQ ID NO: 146 - DNA, 52 bp, unidentified
  source 1..52: mol_type = genomic DNA
ctatgggcgatattgcaaagcaagacgacccgaggcacagccaaattccact

SEQ ID NO: 147 - DNA, 52 bp, unidentified
  source 1..52: mol_type = genomic DNA
ccagggccacagacctggaagtcgccgggagcccggcagcactcctctcaag

SEQ ID NO: 148 - DNA, 52 bp, unidentified
  source 1..52: mol_type = genomic DNA
ctacctgtcggcgggaggcgagcaccgggcgcccgcacgtcgcgccggcggg

SEQ ID NO: 149 - DNA, 52 bp, unidentified
  source 1..52: mol_type = genomic DNA
gggacacctactctgggccccggcacggccgagacagacaggtgcctgcaaa

SEQ ID NO: 150 - DNA, 52 bp, unidentified
  source 1..52: mol_type = genomic DNA
ggccgtcgggcgggggccggctcaccgcggcgcgcgtgggagggccgggccg

SEQ ID NO: 151 - DNA, 52 bp, unidentified
  source 1..52: mol_type = genomic DNA
ccgtcgggcgggggccggctcaccgcggcgcgcgtgggagggccgggccggc

SEQ ID NO: 152 - DNA, 52 bp, unidentified
  source 1..52: mol_type = genomic DNA
ttccaagggcgccgggccgcctccgcgcgtggagcgtccactgttggggtaa

SEQ ID NO: 153 - DNA, 52 bp, unidentified
  source 1..52: mol_type = genomic DNA
caccctccggacaccccagggagcacggacaggtcttcccagcacggcaggc

SEQ ID NO: 154 - DNA, 52 bp, unidentified
  source 1..52: mol_type = genomic DNA
gtgagacggaatgtgtgagtatgtacgtaacaggttttaagaaattgtgtac

SEQ ID NO: 155 - DNA, 52 bp, unidentified
  source 1..52: mol_type = genomic DNA
gagctcttcaagggcttcgtcaacgcggtcgccgcggtgcgccaggaccccg

SEQ ID NO: 156 - DNA, 52 bp, unidentified
  source 1..52: mol_type = genomic DNA
tcaagggcttcgtcaacgcggtcgccgcggtgcgccaggaccccgacggcac

SEQ ID NO: 157 - DNA, 52 bp, unidentified
  source 1..52: mol_type = genomic DNA
tttgggtcttagccaaggtacacatcggcgtttgttttgcatttgtgatcta

SEQ ID NO: 158 - DNA, 52 bp, unidentified
  source 1..52: mol_type = genomic DNA
gacaagggtatggaatacctacaaacggtgattctgattcttttcaagggtg

SEQ ID NO: 159 - DNA, 52 bp, unidentified
  source 1..52: mol_type = genomic DNA
acaggcaacatcgtgggccttctaccgggcacttccttcttctctgcccgca

SEQ ID NO: 160 - DNA, 52 bp, unidentified
  source 1..52: mol_type = genomic DNA
gcccagaggtatccacagcccagctcgctgtggtctcacacacagcgtctca

SEQ ID NO: 161 - DNA, 52 bp, unidentified
  source 1..52: mol_type = genomic DNA
cggctgccgcgcccgcagtcgtcgccgccgcgctcctgccaggccgacctgg

SEQ ID NO: 162 - DNA, 52 bp, unidentified
  source 1..52: mol_type = genomic DNA
cgtcgccgccgcgctcctgccaggccgacctggagcggaagctgaagccgaa

SEQ ID NO: 163 - DNA, 52 bp, unidentified
  source 1..52: mol_type = genomic DNA
gcgctcctcgccgctctcggtggcccggagaacaaaagccccgggcctggcc

SEQ ID NO: 164 - DNA, 52 bp, unidentified
  source 1..52: mol_type = genomic DNA
ggagggcagctgggagtacttcccacgcccacatgagcctttgtgtgaaaat

SEQ ID NO: 165 - DNA, 52 bp, unidentified
  source 1..52: mol_type = genomic DNA
aattactttcactttgttgtgaggtcgctgctgtcttctgtgtcaccccagg

SEQ ID NO: 166 - DNA, 52 bp, unidentified
  source 1..52: mol_type = genomic DNA
ctggtgcccgacggaaataggaggccggcagcctgccagagtgggacagtcc

SEQ ID NO: 167 - DNA, 52 bp, unidentified
  source 1..52: mol_type = genomic DNA
gtggccctgttaccctggtgcccgacggaaataggaggccggcagcctgcca

SEQ ID NO: 168 - DNA, 52 bp, unidentified
  source 1..52: mol_type = genomic DNA
gggggtcgcttggcttggttgcccgcgaagaaggagttggtgtcctgcaggc

SEQ ID NO: 169 - DNA, 52 bp, unidentified
  source 1..52: mol_type = genomic DNA
gggcggggcctcccgggctggagtgcggtacctgagcgagacgcgcaccaga

SEQ ID NO: 170 - DNA, 52 bp, unidentified
  source 1..52: mol_type = genomic DNA
acacgccgctgcgctccttggacgacgacacggcctggacgatcagctccga

SEQ ID NO: 171 - DNA, 52 bp, unidentified
  source 1..52: mol_type = genomic DNA
gctgacgccgtccttcacgcgcgggcggcacgtgtggaaaacgcagcgcgca

SEQ ID NO: 172 - DNA, 52 bp, unidentified
  source 1..52: mol_type = genomic DNA
cccggtggtctccaacacagccaggcgtcgggttttgtgcgggatggtgcag

SEQ ID NO: 173 - DNA, 52 bp, unidentified
  source 1..52: mol_type = genomic DNA
ttcatcattggatgatagtttttcacgaccttgagagtctccttgtaaaact

SEQ ID NO: 174 - DNA, 52 bp, unidentified
  source 1..52: mol_type = genomic DNA
cccttttttacacatgaacaaaacgcgctacagagaggaataacctccccag

SEQ ID NO: 175 - DNA, 52 bp, unidentified
  source 1..52: mol_type = genomic DNA
gccacgcggcccgcgcccagccccacgcacagcgacagcaggtccgacagca

SEQ ID NO: 176 - DNA, 52 bp, unidentified
  source 1..52: mol_type = genomic DNA
ggagtctaggatggcagtgtgactccgagaaggtaggacagcgcgggagagg

SEQ ID NO: 177 - DNA, 52 bp, unidentified
  source 1..52: mol_type = genomic DNA
cctaaataaactcttaggaattccccggttccttctccctgtccccactgca

SEQ ID NO: 178 - DNA, 52 bp, unidentified
  source 1..52: mol_type = genomic DNA
cgcccgaccccggaacccccggtggcgtcactgctgcgcgtccgccggagca

SEQ ID NO: 179 - DNA, 52 bp, unidentified
  source 1..52: mol_type = genomic DNA
atggcctcctgtctatcctgggacccgtctccacccccatcagagccaatga

SEQ ID NO: 180 - DNA, 52 bp, unidentified
  source 1..52: mol_type = genomic DNA
tgcgccccggggtctggctcgtccgcgcggggcagcggcccatggtcggcgg

SEQ ID NO: 181 - DNA, 52 bp, unidentified
  source 1..52: mol_type = genomic DNA
tattacagtgtcgtcgagtcaaacgcgcttttcactcaatggaaattcctct

SEQ ID NO: 182 - DNA, 52 bp, unidentified
  source 1..52: mol_type = genomic DNA
gcgggctgggggcccggaggggccgcgcctcgcaccgcggacgctgcagttc

SEQ ID NO: 183 - DNA, 52 bp, unidentified
  source 1..52: mol_type = genomic DNA
gggagggcggctgctcgggtcactccgggggcagcatgcgcggcggcgctgg

SEQ ID NO: 184 - DNA, 52 bp, unidentified
  source 1..52: mol_type = genomic DNA
gtgtcgtgcacgttagcgtggccgacggcgtggtgggccctgggccgcaagc

SEQ ID NO: 185 - DNA, 52 bp, unidentified
  source 1..52: mol_type = genomic DNA
tagcagggctctgggagtccctagccggtgcaaagtgttagaaagacatcac

SEQ ID NO: 186 - DNA, 52 bp, unidentified
  source 1..52: mol_type = genomic DNA
ttgagctgcctggagcagcagccgtcggccacctagcaggagtgtcctcgca

SEQ ID NO: 187 - DNA, 52 bp, unidentified
  source 1..52: mol_type = genomic DNA
acacgatagcccctggcccggtccccggccacccccgccagcagcccgatgc

SEQ ID NO: 188 - DNA, 52 bp, unidentified
  source 1..52: mol_type = genomic DNA
acttccttggtcagaaccacggcttcgcctgagaactaattgttaccctcga

SEQ ID NO: 189 - DNA, 52 bp, unidentified
  source 1..52: mol_type = genomic DNA
ggtatcccgtcccctcggctcggctcggctcggctcggctcggcgggccccg

SEQ ID NO: 190 - DNA, 52 bp, unidentified
  source 1..52: mol_type = genomic DNA
ccagctttgtttgggtctcccacaccgcctgtgtatgaatgggactaaattt

SEQ ID NO: 191 - DNA, 52 bp, unidentified
  source 1..52: mol_type = genomic DNA
cgtacagcgcgtcctgcgcctccagcggcatggccgggccgaccggccccgg

SEQ ID NO: 192 - DNA, 52 bp, unidentified
  source 1..52: mol_type = genomic DNA
acgtaaggtgagaaggcgcgagcgccgcggacccgtccggtgggatgagggg

SEQ ID NO: 193 - DNA, 52 bp, unidentified
  source 1..52: mol_type = genomic DNA
tcctcggccgtcagcaccgtctggccggacatgtcgctggcgctgccgccgc

SEQ ID NO: 194 - DNA, 52 bp, unidentified
  source 1..52: mol_type = genomic DNA
gaccctgtgccctcagctggcctcacgtgcatggaatggtgctgtgccctgt

SEQ ID NO: 195 - DNA, 52 bp, unidentified
  source 1..52: mol_type = genomic DNA
ctgccgggcctggccggggccgcttcgtggtctgcacttaccccacgtgcca

SEQ ID NO: 196 - DNA, 52 bp, unidentified
  source 1..52: mol_type = genomic DNA
taaaggggcgatgccgcgacccaggcgctggcgtggggtccaccggcgtcgc

SEQ ID NO: 197 - DNA, 52 bp, unidentified
  source 1..52: mol_type = genomic DNA
agagtgtagagctgctgggggctgccgtctgccctgctggcctgctgtggta

SEQ ID NO: 198 - DNA, 52 bp, unidentified
  source 1..52: mol_type = genomic DNA
ttcatctctggttcttgtcaatccccgcaggaactgtgtccttcagacacag

SEQ ID NO: 199 - DNA, 52 bp, unidentified
  source 1..52: mol_type = genomic DNA
gtgaaattcctgcaagtcgtgttgccgacatagtctgcaggacaaactttta

SEQ ID NO: 200 - DNA, 52 bp, unidentified
  source 1..52: mol_type = genomic DNA
gagaggcgggcccctggcttttgggcgcagacctccagggcccgcatcaagt

SEQ ID NO: 201 - DNA, 52 bp, unidentified
  source 1..52: mol_type = genomic DNA
tccagaccctagcagttggaggatccgagttcgaattctgtctttgccattc

SEQ ID NO: 202 - DNA, 52 bp, unidentified
  source 1..52: mol_type = genomic DNA
tgcacgaccaccgcagcgtcccaaccgtggtcctggcccctggctccctcct

SEQ ID NO: 203 - DNA, 52 bp, unidentified
  source 1..52: mol_type = genomic DNA
gcggcagaagtccggcgtgggcgcccgggtcgggggcgcgtccgagaccccc

SEQ ID NO: 204 - DNA, 52 bp, unidentified
  source 1..52: mol_type = genomic DNA
ggcccgattgtgccgcctgttgtgtcggttgactcttgcccgattgccgcgc

SEQ ID NO: 205 - DNA, 52 bp, unidentified
  source 1..52: mol_type = genomic DNA
cctggtgccggagagcgggatgtcacggcttgtatcgatcacgcgttgaacc

SEQ ID NO: 206 - DNA, 52 bp, unidentified
  source 1..52: mol_type = genomic DNA
ggccgtccatggcagtgccggcccacgaggcagggggcagataagggggccg

SEQ ID NO: 207 - DNA, 52 bp, unidentified
  source 1..52: mol_type = genomic DNA
tctctggcgcaataaaagagacccccgtgaggaagcccccccagcactcata

SEQ ID NO: 208 - DNA, 52 bp, unidentified
  source 1..52: mol_type = genomic DNA
gagaaggacgtccctgtcgcgaagccgttcgcagcgcgcggccctgggggcg

SEQ ID NO: 209 - DNA, 52 bp, unidentified
  source 1..52: mol_type = genomic DNA
gatagcttcctcggctcggtcggggcggctgctgagcggcggagcggcgcgg

SEQ ID NO: 210 - DNA, 52 bp, unidentified
  source 1..52: mol_type = genomic DNA
gaggggacctgccccgtggggtggccgtgcaggttcgggggcgcatcctgcg

SEQ ID NO: 211 - DNA, 52 bp, unidentified
  source 1..52: mol_type = genomic DNA
cagggacctcggccctccctcccagcggccgggaccagactccggggtgcct

SEQ ID NO: 212 - DNA, 52 bp, unidentified
  source 1..52: mol_type = genomic DNA
gcgtccccggaggtgtttaccgacccgcgcacacacaccccgctacgtgcca

SEQ ID NO: 213 - DNA, 52 bp, unidentified
  source 1..52: mol_type = genomic DNA
agggctgggaagattggcagcccttcggagtttatgcgtgaagggagtccat

SEQ ID NO: 214 - DNA, 52 bp, unidentified
  source 1..52: mol_type = genomic DNA
gattggcagcccttcggagtttatgcgtgaagggagtccatcttaccctccc

SEQ ID NO: 215 - DNA, 52 bp, unidentified
  source 1..52: mol_type = genomic DNA
ccacagcatcagcccaggagaggcacggtttattgacaggttacaccccagg

SEQ ID NO: 216 - DNA, 52 bp, unidentified
  source 1..52: mol_type = genomic DNA
ccacagcggaaaccatcactggcaccggctactccttacggacaatgctggg

SEQ ID NO: 217 - DNA, 52 bp, unidentified
  source 1..52: mol_type = genomic DNA
aaatgtacagccagcgatttggcatcgtacagcgggaggttaagggccccac

SEQ ID NO: 218 - DNA, 52 bp, unidentified
  source 1..52: mol_type = genomic DNA
acggagaaaccactttatctgaggacggtcgaaaattagatcctactttccc

SEQ ID NO: 219 - DNA, 52 bp, unidentified
  source 1..52: mol_type = genomic DNA
attttagatgccaattctttgaaggcgttggatgtaacaccccttcatttcc

SEQ ID NO: 220 - DNA, 52 bp, unidentified
  source 1..52: mol_type = genomic DNA
ggcgggcaggtagagggccgatccccggccttttcacatcccacttctttcc

SEQ ID NO: 221 - DNA, 52 bp, unidentified
  source 1..52: mol_type = genomic DNA
acccttgccctccttggcaggccagcgctccgggaggccctaaagaacggac

SEQ ID NO: 222 - DNA, 52 bp, unidentified
  source 1..52: mol_type = genomic DNA
gtgtttacctggagactaattcatacggtagttattgggctttaaatccagg

SEQ ID NO: 223 - DNA, 52 bp, unidentified
  source 1..52: mol_type = genomic DNA
gtggccagcctatttagttccacatcgttcagcctagatgccccccaccact

SEQ ID NO: 224 - DNA, 52 bp, unidentified
  source 1..52: mol_type = genomic DNA
tctgtaagagtgtttcccttcgacacggtgtatttctggaaggttttcttct

SEQ ID NO: 225 - DNA, 52 bp, unidentified
  source 1..52: mol_type = genomic DNA
gggccagtgagtgcggctgcgcggccgggagggcggcgggcggcccgcgggc

SEQ ID NO: 226 - DNA, 52 bp, unidentified
  source 1..52: mol_type = genomic DNA
tcgccgtcgctggagcaggcggagtcggacgcgctggacatcagcaccaaag

SEQ ID NO: 227 - DNA, 52 bp, unidentified
  source 1..52: mol_type = genomic DNA
gaggtcagcatgcgcctccactccccgcgagtggacttggagcggctcagca

SEQ ID NO: 228 - DNA, 52 bp, unidentified
  source 1..52: mol_type = genomic DNA
gcgtggcgcccgaagcgctgccactcgcgcgccagcttgcgccccttctgcc

SEQ ID NO: 229 - DNA, 52 bp, unidentified
  source 1..52: mol_type = genomic DNA
atttcctgagtgcccactctgtgcccgacatgggtcacacagctcccccagg

SEQ ID NO: 230 - DNA, 52 bp, unidentified
  source 1..52: mol_type = genomic DNA
gagcccaggccgccgtgccagcccacgtcaccgcagacatacacagacacgc

SEQ ID NO: 231 - DNA, 52 bp, unidentified
  source 1..52: mol_type = genomic DNA
cacctgtgctagtccagcctgatggcgcaggtgcagcccaacactcaggggg

SEQ ID NO: 232 - DNA, 52 bp, unidentified
  source 1..52: mol_type = genomic DNA
aggaagggggggcgagggcatgcttcgagtttggatccgaggtcttcagggg

SEQ ID NO: 233 - DNA, 52 bp, unidentified
  source 1..52: mol_type = genomic DNA
cgggagcccagcgacccggggaaaccgatgttaagatgaaggtattttcatt

SEQ ID NO: 234 - DNA, 52 bp, unidentified
  source 1..52: mol_type = genomic DNA
gccttccccactcagcggccgggcccggctttcccgccccacggcccggcca

SEQ ID NO: 235 - DNA, 52 bp, unidentified
  source 1..52: mol_type = genomic DNA
ggcgctggacgggcattccggggggcgggggagccagcccgaccccaagcac

SEQ ID NO: 236 - DNA, 52 bp, unidentified
  source 1..52: mol_type = genomic DNA
ttgctgatctctgccccgtggagcccgccccccgaggttcccagttacactc

SEQ ID NO: 237 - DNA, 52 bp, unidentified
  source 1..52: mol_type = genomic DNA
ccgggctaccgggtgcggggggcagcggcgccatcagcacggccacctacac

SEQ ID NO: 238 - DNA, 52 bp, unidentified
  source 1..52: mol_type = genomic DNA
gcgaggctgcgaggctgcgaggctgcgaggcgcggcgccccgtggtggtcac

SEQ ID NO: 239 - DNA, 52 bp, unidentified
  source 1..52: mol_type = genomic DNA
gcctcgcagccctggcgcgcgcgctcgcagatggagcggcacggcgggatgg

SEQ ID NO: 240 - DNA, 52 bp, unidentified
  source 1..52: mol_type = genomic DNA
ctggcgcgcgcgctcgcagatggagcggcacggcgggatggcctgctccagc

SEQ ID NO: 241 - DNA, 52 bp, unidentified
  source 1..52: mol_type = genomic DNA
aagcccctcccccaagccaaggacccggtcacccatcccctccacctcccta

SEQ ID NO: 242 - DNA, 52 bp, unidentified
  source 1..52: mol_type = genomic DNA
cgagtccccctcgcaccccgccccgcggccagtcccgctccggagccctctg

SEQ ID NO: 243 - DNA, 52 bp, unidentified
  source 1..52: mol_type = genomic DNA
gtgaggatgctgctgagcttgcgatcgaaggagccactcatcctggcgagcg

SEQ ID NO: 244 - DNA, 52 bp, unidentified
  source 1..52: mol_type = genomic DNA
gatcgcccggccacatgaccggcgccggccaatcgctggattcaaccactca

SEQ ID NO: 245 - DNA, 52 bp, unidentified
  source 1..52: mol_type = genomic DNA
gagggaccgcgagccgctgattggtcgcaggccgcggtgacgtcggcgggcg

SEQ ID NO: 246 - DNA, 52 bp, unidentified
  source 1..52: mol_type = genomic DNA
ggctccggggcttttccacccgccgcgagtccacttttagtctgtagagaag

SEQ ID NO: 247 - DNA, 52 bp, unidentified
  source 1..52: mol_type = genomic DNA
ccatagcggctgcccagctcgtccccgaagcggaccatggttcccgggcgcc

SEQ ID NO: 248 - DNA, 52 bp, unidentified
  source 1..52: mol_type = genomic DNA
ggaccggggacctgcaggtcggtcacgatgggtggacgtcgctccagccctg

SEQ ID NO: 249 - DNA, 52 bp, unidentified
  source 1..52: mol_type = genomic DNA
gctctgggcctggggactcagcagacgcctccccacccgctagcaggaccct

SEQ ID NO: 250 - DNA, 52 bp, unidentified
  source 1..52: mol_type = genomic DNA
ggaacagctggcctggcctctgcagcgcccaggagctggcccggctgacgcc

SEQ ID NO: 251 - DNA, 52 bp, unidentified
  source 1..52: mol_type = genomic DNA
ccgccatccctgggggtcaagtgcccggcctgtctcctggaggctcgccacg

SEQ ID NO: 252 - DNA, 52 bp, unidentified
  source 1..52: mol_type = genomic DNA
ccggagtgatgacgctgattggctccggccgattttgggggtgatgtcacgg

SEQ ID NO: 253 - DNA, 52 bp, unidentified
  source 1..52: mol_type = genomic DNA
ggtcactgagctctggtttcatcagcgagggaacttactcagctcgagggaa

SEQ ID NO: 254 - DNA, 52 bp, unidentified
  source 1..52: mol_type = genomic DNA
ctgccctgtcccacaagctacctcccgagtgaaatacccatgcgctgagccc

SEQ ID NO: 255 - DNA, 52 bp, unidentified
  source 1..52: mol_type = genomic DNA
cgtgttaccttttgctgggctccttcgcagtcactttgcaggtccttctacc

(19) United States
(12) Patent Application Publication
(10) Pub. No.: US 2025/0346954 A1
(43) Pub. Date: Nov. 13, 2025

(54) METHOD TO IMPROVE HEALTH OUTCOMES OF A DOG USING BIOLOGICAL AGE

(71) Applicant: SOCIÉTÉ DES PRODUITS NESTLÉ S.A., Vevey (CH)

(72) Inventors: LORANE TEXARI, Morges (CH); SEBASTIEN HERZIG, Morges (CH); ALIX ZOLLINGER, Denges (CH); YUANLONG PAN, Chesterfield, MO (US); PHILIPP GUT, Geneve (CH); PASCAL STEINER, St. Louis, MO (US); LAURA LUKJANENKO, Lausanne (CH)

(21) Appl. No.: 18/870,515

(22) PCT Filed: May 30, 2023
(86) PCT No.: PCT/EP2023/064444

§ 371 Date: Nov. 29, 2024

Related U.S. Application Data
(60) Provisional application No. 63/347,106, filed on May 31, 2022.

(51) Int. Cl. C12Q 1/6876

(52) U.S. Cl. CPC C12Q 1/6876; C12Q 2600/154

(57) ABSTRACT

The present invention for selecting a lifestyle regime, dietary regime or therapeutic intervention for a dog, the method comprising: a) providing a DNA methylation profile from a sample obtained from the dog; b) determining a biological age of the dog using the DNA methylation profile; and c) selecting a suitable lifestyle regime, dietary regime or therapeutic intervention for the dog based on the biological age determined in step b).

### FIELD OF THE INVENTION

The present invention relates to a method for determining the biological age of a dog using a DNA methylation profile, in particular the invention relates to methods of selecting a lifestyle regime, dietary regime or therapeutic intervention for the dog, or determining the efficacy of a lifestyle regime, dietary regime or therapeutic intervention, or developing anti-aging lifestyles or dietary regimes based on a biological age determined from the DNA methylation profile.

### BACKGROUND TO THE INVENTION

The ability to determine information regarding the health of a dog is desirable to inform about the dog's general health and well-being.

Chronological age is known to be a major indicator of general health status, with increasing chronological age associated with reduced health. However, depending on genetics, nutrition, and lifestyles, individuals may age slower or faster than their chronological age. Chronological age may therefore not always reflect an individual's rate of aging or risk of reduced health. On the other hand, the biological age of an individual (based on e.g. clinical biochemistry and cell biology measures) can vary compared to others of the same chronological age. Methods for determining biological age may be helpful for identifying individuals at risk of age-related disorders earlier than would be expected based on their chronological age (see e.g. WO2019/046725).

However, there is a need for further methods of determining the biological age of a dog and utilising measures of biological age to improve health outcomes for a dog.

### SUMMARY OF THE INVENTION

In a first aspect, the present invention provides a method for selecting a lifestyle regime, dietary regime or therapeutic intervention for a dog, the method comprising:
a) providing a DNA methylation profile from a sample obtained from the dog;
b) determining a biological age of the dog using the DNA methylation profile; and
c) selecting a suitable lifestyle regime, dietary regime or therapeutic intervention for the dog based on the biological age determined in step b).

Accordingly, the present method enables a suitable lifestyle regime, dietary regime or therapeutic intervention to be selected for the dog, based on its biological age as determined from the DNA methylation profile. For example, highly digestible and high-quality protein diets are generally recommended based upon the chronological age of a dog. For example, it may be recommended that a dog is switched to a senior diet around 7 or 8 years old. However, in the context of the present invention, the determination of an increased biological age for a dog compared to its chronological age may allow a determination to switch the dog to a senior diet at an earlier age. In contrast, a dog with a reduced biological age compared to its chronological age may be able to stay on an adult diet for longer.

The present methods may comprise determining the probability of a lifespan, health span and/or longevity of a dog based on its biological age as determined from the DNA methylation profile; compared to an equivalent dog of—for example—the same chronological age, sex and breed.

As used herein, ‘lifespan’ may refer to the length of time (e.g. years) for which a subject lives. ‘Health span’ may refer to length of time (e.g. years) of life without disease. ‘Longevity’ may refer to length of time (e.g. years) that a subject lives beyond its expected lifespan.

Suitably, the present methods may comprise selecting and/or applying a lifestyle regime, dietary regime or therapeutic intervention to a dog following a determination that the dog's biological age is greater than its chronological age.

As used herein, ‘selecting a suitable lifestyle regime, dietary regime or therapeutic intervention for a dog’ may also encompass ‘recommending a lifestyle regime, dietary regime or therapeutic intervention for the dog’ or ‘providing a recommended lifestyle regime, dietary regime or therapeutic intervention for the dog’.

In another aspect, the present invention provides a method for determining the efficacy of a lifestyle regime, dietary regime or therapeutic intervention for improving the biological age of a dog, said method comprising:
a) applying a lifestyle regime, dietary regime or therapeutic intervention to the dog, wherein the lifestyle regime, dietary regime or therapeutic intervention has been selecting according to the first aspect of the invention;
b) after a time period of applying the lifestyle regime, dietary regime or therapeutic intervention to the dog; determining a biological age of the dog using a DNA methylation profile from a sample obtained from the dog;
c) determining if there has been a change in the biological age of the dog after the time period of following the lifestyle regime, dietary regime or therapeutic intervention.

In another aspect, the invention provides a method for determining the efficacy of a lifestyle regime, dietary regime or therapeutic intervention for improving the biological age of a dog, said method comprising:
a) determining a biological age of the dog using a DNA methylation profile from a sample obtained from the dog;
b) applying a lifestyle regime, dietary regime or therapeutic intervention selected based on the biological age determined in step a) to the dog;
c) after a time period of applying the lifestyle regime, dietary regime or therapeutic intervention to the dog; determining a biological age of the dog using a DNA methylation profile from a sample obtained from the dog;
d) determining if there has been a change in the biological age of the dog between step a) and step c).

In a further aspect, the invention relates to a method for determining the efficacy of a lifestyle regime, dietary regime or therapeutic intervention for improving the biological age of a dog, said method comprising:
a) determining a biological age of the dog using a DNA methylation profile from a sample obtained from the dog;
b) applying a lifestyle regime, dietary regime or therapeutic intervention to the dog;
c) after a time period of applying the lifestyle regime, dietary regime or therapeutic intervention to the dog; determining a second biological age of the dog using a DNA methylation profile from a second sample obtained from the dog;
d) determining if there has been a change in the first and second biological age of the dog after the time period of following the lifestyle regime, dietary regime or therapeutic intervention.

Suitably, improving the biological age of a dog may refer to a reduction in the difference between the biological age and chronological age of the dog, where the biological age of the dog is greater than its chronological age. Further, improving the biological age of a dog may refer to maintaining or further increasing the difference between the biological age and chronological age of the dog, where the biological age of the dog is less than its chronological age. Alternatively, a worsening in the biological age of a dog may refer to an increase in the difference between the biological age and chronological age of the dog, where the biological age of the dog is greater than its chronological age. A worsening in the biological age of a dog may also refer to a decrease in the difference between the biological age and chronological age of the dog, where the biological age of the dog is less than its chronological age.

Suitably, improving the biological age of a dog may refer to a reduction in the rate of change between the biological age and chronological age of the dog, where the biological age of the dog is greater than its chronological age. For example, a dog's biological age may have been increasing by 1.5 years per 1 year increase in chronological age. Following a lifestyle and dietary regime intervention, a reduction in the rate of change such that the dog's biological age subsequently increases by 1.25 years per 1 year increase in chronological age may provide an improvement in the dog's biological age.

Improving the biological age may also refer to maintaining or increasing in the rate of change between the biological age and chronological age of the dog, where the biological age of the dog is less than its chronological age. For example, a dog's biological age may have been increasing by less than 1 year (e.g 0.9 years) per 1 year increase in chronological age. Following a lifestyle, dietary regime or therapeutic intervention, the rate of change may alter such that the dog's biological age subsequently increases by, for example, 0.8 years or fewer per 1 year increase in chronological age may provide an improvement in the dog's biological age.

The present methods for determining the efficacy of a lifestyle regime, dietary regime or therapeutic intervention for improving the biological age of a dog may advantageously allow ongoing monitoring of the effectiveness of a lifestyle regime, dietary regime or therapeutic intervention for improving or maintaining the health of the dog. The use of such methods may advantageously allow particularly effective lifestyle regime, dietary regime or therapeutic interventions to be identified. In contrast, if a lifestyle regime, dietary regime or therapeutic intervention is determined to be ineffective based on the biological age of the dog; an alternative lifestyle regime, dietary regime or therapeutic intervention may then be implemented.

In another aspect the invention provides a method for preventing or reducing the risk of a dog developing a disease; the method comprising:
a) determining a biological age of the dog using a DNA methylation profile from a sample obtained from the dog; wherein the biological age determined for the dog is associated with an increased likelihood to develop the disease; and
b) selecting a lifestyle regime, dietary regime or therapeutic intervention for the dog based on the biological age determined in step a);
wherein the lifestyle regime, dietary regime or therapeutic intervention prevents or reduces the risk of the dog developing the disease.

Suitably, the disease is an age-related disease. For example, the age-related disease may be osteoarthritis, dementia, cognitive dysfunction, pre-diabetic condition, diabetes, cancer, heart disease, obesity, gastrointestinal disorders, incontinence, kidney disease, sarcopenia, vision loss, hearing loss, osteoporosis, cataracts, cerebrovascular disease, and/or liver disease.

The method may optionally further comprise administering the lifestyle regime, dietary regime or therapeutic intervention to the dog. Suitably, the lifestyle regime may be a dietary intervention or a therapeutic modality.

In another aspect, the invention relates to a method for selecting a dog as being suitable for receiving an anti-aging lifestyle regime, dietary regime or therapeutic intervention; the method comprising: a) determining a biological age of the dog using a DNA methylation profile from a sample obtained from the dog; b) selecting the dog as being suitable for receiving an anti-aging lifestyle regime, dietary regime or therapeutic intervention if it has an increased biological age compared to its chronological age.

Suitably, whilst an anti-aging lifestyle regime, dietary regime or therapeutic intervention may be effective for dogs based on chronological age, it may be particularly effective when applied to a dog with an increased biological age compared to its chronological age. As such, the present method may advantageously enable the selection of a dog that has an increased likelihood to respond, or improved magnitude of response, to the anti-aging lifestyle regime, dietary regime or therapeutic intervention.

The lifestyle regime, dietary regime or therapeutic intervention may be selected based on a determination that the dog has an increased biological age compared to its chronological age.

The lifestyle regime, dietary regime or therapeutic intervention may be a dietary intervention. The dietary intervention may be a calorie-restricted diet, a senior diet or a low protein diet.

The DNA methylation profile may be associated with increased biological age of (i) a tissue; (ii) an organ; or (iii) a physiological system, such as the immune, gastrointestinal, urinary, muscular, cardiovascular, and/or neurological system.

The invention further provides a dietary intervention for use in improving the biological age of a dog, wherein the dietary intervention is administered to a dog with a biological age determined by the method of the invention.

The invention further relates to the use of a dietary intervention to improve the biological age of a dog, wherein the dietary intervention is administered to a dog with a biological age determined by the method of the invention.

The invention further provides a computer-readable medium comprising instructions that when executed cause one or more processors to perform the method of the invention.

The invention also provides a computer system for selecting a suitable lifestyle regime, dietary regime or therapeutic intervention for a dog, the computer system programmed to perform one or more of the steps of: a) determining a biological age of the dog using a DNA methylation profile from the dog; and b) selecting a suitable lifestyle regime, dietary regime or therapeutic intervention for the dog based on the biological age determined in step a).

In another aspect, the invention provides a computer system for determining the efficacy of a lifestyle regime, dietary regime or therapeutic intervention for improving the biological age of a dog, the computer system programmed to perform one or more of the steps of: a) determining a biological age of the dog using a DNA methylation profile from a sample obtained from the dog before the lifestyle regime, dietary regime or therapeutic intervention and a sample obtained from the dog after the lifestyle regime, dietary regime or therapeutic intervention; and b) determining if there has been a change in the biological age of the dog between the sample obtained from the dog before and after the lifestyle regime, dietary regime or therapeutic intervention has been applied.

In a further aspect the invention provides a computer system for determining a likelihood that a dog will benefit from an anti-aging lifestyle regime, dietary regime or therapeutic intervention; the computer system programmed to perform one or more of the steps of: a) determining a biological age of the dog using a DNA methylation profile from a sample obtained from the dog; b) identifying a dog as likely to respond to an anti-aging lifestyle regime, dietary regime or therapeutic intervention if it has an increased biological age compared to its chronological age.

In a further aspect the invention provides a computer program product comprising computer implementable instructions for causing a programmable computer to determine a biological age of the dog using a DNA methylation profile from the dog; and select a suitable lifestyle regime, dietary regime or therapeutic intervention for the dog based on the biological age determined using a DNA methylation profile.

In another aspect the invention provides a computer program product comprising computer implementable instructions for causing a programmable computer to a) determine a biological age of a dog using a DNA methylation profile from a sample obtained from the dog before a lifestyle regime, dietary regime or therapeutic intervention and a sample obtained from the dog after the lifestyle regime, dietary regime or therapeutic intervention; and b) determine if there has been a change in the biological age of the dog between the sample obtained from the dog before and after the lifestyle regime, dietary regime or therapeutic intervention has been applied.

In a further aspect the invention provides a computer program product comprising computer implementable instructions for causing a programmable computer to a) determine a biological age of a dog using a DNA methylation profile from a sample obtained from the dog; b) identify a dog as likely to respond to an anti-aging lifestyle regime, dietary regime or therapeutic intervention if it has an increased biological age compared to its chronological age.

Advantageously, the present invention may allow a biological age to be determined based on markers of multiple organ systems and functions. Accordingly, the present methods may advantageously encompassed a range of potential organ dysfunctions.

Evaluating the biological age of a dog allows one to test several aspects of the animal's wellbeing. First, it can predict whether this animal is more likely to need a dietary or supplement-based intervention. It can also be used to test the efficacy of a dietary or supplement-based intervention on aging.

### FIGURES

FIG. 1—Biological age predicted using an illustrative biological clock of the present invention is highly correlated with chronological age

FIG. 2—Validation of an illustrative biological clock of the present invention in a calorie restriction study. Delta corresponds to the residuals of the regression model of Chronological Age vs predicted biological age using the first gen clock. Dogs on a calorie restriction have a significantly lower biological age (lower delta) compared to dogs on a control diet.

FIG. 3—An illustrative biological clock comprising the top 3 methylation sites from the full biological clock correlates with chronological age

FIG. 4—An illustrative biological clock comprising the top 5 methylation sites from the full biological clock correlates with chronological age

FIG. 5—An illustrative biological clock comprising the top 10 methylation sites from the full biological clock correlates with chronological age

FIG. 6—An illustrative biological clock comprising the top 20 methylation sites from the full biological clock correlates with chronological age

FIG. 7—An illustrative biological clock comprising the top 50 methylation sites from the full biological clock correlates with chronological age

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

The methods and systems disclosed herein can be used by veterinarians, health-care professionals, lab technicians, pet care providers and so on.

### Subject

The present methods are directed to canine subjects. Accordingly, the subject of the present invention is a dog.

In an alternative aspect, the subject may be a feline subject. Accordingly, in the alternative aspects of the invention, the subject is a cat. All disclosures herein are equally applicable to a cat, unless stated otherwise.

### Breed

The present methods may utilise information regarding the breed of the dog. The dog may be categorised as a toy, small, medium, large or giant breed—for example. Suitably, the dog breed may be categorised based on the weight of the dog. Suitably, the dog breed may be categorised based on the average weight of a dog for a given breed.

Suitably, the dog may be categorised as a small or medium breed. Suitably, the categorisation is determined by the average weight of adult dogs of this breed. Suitably, a breed with an average weight below 10 kg is categorised as a small breed and/or a breed with an average weight above 10 kg is categorised as a medium breed.

In the alternative aspect where the subject is a cat, the cat may be a domestic cat. Suitably, the cat may be a Domestic Shorthair cat.

### Sex

Suitably, the sex of the dog may be classified as male or female.

### Chronological Age

Chronological age may be defined as the amount of time that has passed from the subject's birth to the given date. Chronological age may be expressed in terms of years, months, days, etc.

Suitably, the present method may be applied to a dog of any chronological age. In certain embodiments, the dog may be at least about 2 years old. Suitably, the dog may be at least about 2, at least about 3, at least about 4, at least about 5, at least about 6, at least about 7, at least about 8, at least about 9 or at least about 10 years old.

Suitably, the dog may be at least about 7 years old.

### Sample

The present invention comprises a step of providing or determining a DNA methylation profile from one or more samples obtained from a subject.

Suitably, the sample is a blood, hair follicle, buccal swab, saliva or tissue sample.

Suitably, the sample is a hair follicle, buccal swab or saliva sample. Such sample types are particularly applicable if the sample is to be provided, for example, outside of a veterinarian environment—for example using a kit according to the present invention.

Suitably, the sample is derived from blood. The sample may contain a blood fraction or may be whole blood. The sample preferably comprises whole blood. The sample may comprise a peripheral blood mononuclear cell (PBMC) or lymphocyte sample. Techniques for collecting samples from a subject and extracting DNA (e.g. genomic DNA) from the sample are well known in the art.

The present methods may be performed on one or more samples obtained from the subject. For example, the method may be performed using a first sample obtained at a given time point and a second sample obtained following a time interval after the first sample was obtained. The method may be performed more than once, on samples obtained from the same dog over a time period. For example, samples may be obtained repeatedly once per month, once a year, or once every two years. Suitably, the samples may be obtained around once per year (e.g. during an annual veterinary health check). This may be useful in determining the effects of a particular treatment or change in lifestyle—such as a dietary intervention or a change in exercise regime.

In one embodiment, the method may be applied to a sample obtained from a subject prior to a change in lifestyle (e.g. a dietary product intervention or a change in exercise regime). In another embodiment, the method may be applied to a sample obtained from a subject prior to, and after the e.g. dietary product intervention or change in exercise regime. The method may also be applied to samples obtained at predetermined times throughout the e.g. dietary product intervention or change in exercise regime. These predetermined times may be periodic throughout the e.g. dietary product intervention or change in exercise regime, e.g. every day or three days, or may depend on the subject being tested.

### DNA Methylation

DNA methylation is the process by which a methyl group (CH₃) is added covalently to a cytosine base that is part of a DNA molecule. In vivo, this process is catalysed by a family of DNA methyltransferases (Dnmts), that generate the modified cytosine by transfer of a methyl group from S-adenyl methionine (SAM). The cytosine is modified on the 5^{th} carbon atom, and the modified residue is known as 5-methylcytosine (5mC). The DNA methylation may also comprise 5-hydroxymethylcytosine (5hmc).

DNA methylation is an example of an epigenetic mechanism, i.e. it is capable of modifying gene expression without modification of the underlying DNA sequence. DNA methylation can, for example, inhibit the expression of genes by acting as a recruitment signal for repressive factors, or by directly blocking transcription factor recruitment. DNA methylation predominantly occurs in the genome of somatic mammalian cells at sites of adjacent cytosine and guanine that form a dinucleotide (CpG). While non-CpG methylation is observed in embryonic development, in the adult these modifications are much reduced in most cell types. CpG islands are stretches of DNA that have a high CpG density, but are generally unmethylated. These regions are associated with promoter regions, particularly promoter regions of housekeeping genes, and are thought to be maintained in a permissive state to allow gene expression.

DNA methylation has been found to vary with age in humans and other animals. Aged mammalian tissues show overall DNA hypomethylation, which is considered to be due to a gradual loss or mis-targeting of DMNT1 methyltransferase activity, but local hypermethylation of CpG islands. Local hypermethylation can result in repression of certain genes and this can contribute towards age-related disease. The link between epigenetic changes in DNA methylation with age allows the estimation of a “biological age” using “DNA methylation clocks”. These clocks are usually trained against chronological age using supervised machine learning approaches, and deviations of the “clock age” from the actual chronological age for an individual is considered an indicator of “biological” age. This correlates with the chronological age of the individual, but deviations from correlation can indicate potential risk of age-related disease or illness in individuals.

The detection of specific methylated DNA can be accomplished by multiple methods (see e.g. Zuo et al., 2009; Epigenomics. 1(2):331-345) and Rauluseviciute et al.; Clinical Epigenetics; 2019; 11(193)). A number of methods are available for detection of differentially methylated DNA at specific loci in samples such as blood, urine, stool or saliva. These methods are able to distinguish 5-methyl cytosine or methylated DNA from unmethylated DNA, and subsequently quantify the proportion of methylated and unmethylated DNA for a particular genomic site.

The present methods may comprise determining a DNA methylation profile for dog using any suitable method. Suitable methods include, but are not limited to, those described below.

### Enzymatic Methyl-seq (EM-seq)

Suitably, enzymatic approaches are used to detect 5mC and 5hmC. By way of example, Enzymatic Methyl-seq (EM-seq) may be used.

Typically in EM-seq, in a first enzymatic step, 5mC is oxidized to 5hmC, then 5fC and finally 5caC by the activity of Tet methylcytosine dioxygenase 2 (TET2). In addition, use of a T4-BGT enzyme glucosylates both the pre-existing 5hmC and that produced by TET2 activity. In a second enzymatic step, following denaturation of the double-stranded DNA, the enzyme apolipoprotein B mRNA editing enzyme catalytic polypeptide-like 3A (APOBEC3A) is used to deaminate cytosines, but is unable to deaminate the oxidised or glycosylated forms of 5mC and 5hmC. Only unmethylated cytosines are deaminated to form uracil bases. Prior to the first enzymatic step, the DNA fragments may be generated from mechanical shearing and end-repaired, A-tailed, and ligated to sequencing adaptors, which can be carried out using the NEBNext® DNA Ultra II reagents (NEB), for example. Following the second enzymatic step, the deaminated single-stranded DNA may be amplified by PCR reactions, using polymerase such as NEBNext® Q5U™ which can amplify uracil containing templates, and the resulting library can be sequenced or analysed in an identical manner to the DNA sample generated by bisulfite sequencing. The output of EM-seq is generally the same as whole genome bisulfite sequencing, but with the use of less DNA-damaging reagents, which consequently reduces sample loss, and can outperform bisulfite-conversion prepared samples in coverage, sensitivity and accuracy of cytosine methylation calling. An illustrative EM-seq method is described by Vaisvila et al. (Genome Research; 2021; 31:1-10).

### Bisulfite Conversion-Based Methods

Bisulfite conversion utilizes the selective conversion of unmethylated cytosines to uracil when treated with sodium bisulfite. Denatured DNA is treated with sodium bisulfite, which converts all unmodified cytosines to uracil, and subsequent PCR amplification converts these residues to thymines. Analysing the produced DNA sequences can be done via many different methods, examples of which include but are not limited to: denaturing gel electrophoresis, single-strand conformation polymorphism, melting curves, fluorescent real-time PCR (MethyLight), MALDI mass spectrometry, array hybridization, and sequencing (e.g. Whole Genome Bisulfite Sequencing WGBS). Recently developed techniques such as SeqCap Epi enrich sequences of interest prior to sequencing that enables deeper coverage over a more focused area). Comparison of the abundance of sequences in a bisulfite-converted sample against those of an untreated control allows analysis of methylation at a target site, where the proportion of converted sequences is indicative of the level of methylation at the target site.

Further variants of the bisulfite conversion method are available that are able to distinguish 5mC from the oxidised form 5-hydroxymethylcytosine (5hmC), which behaves identically to 5mC under standard bisulfite conversion, and to detect the further modification 5-formylcytosine (5fC). These methods, such as oxBS-Seq and redBS-Seq, utilise oxidation and reduction of these markers to modify the susceptibility of each species to bisulfite conversion, and through comparative analysis quantify the amount of each modification at target loci.

### Selective Restriction Endonuclease Digestion Methods

Methods of analysing DNA methylation patterns exist may involve the use of restriction enzymes. These include, for example, restriction landmark genomic scanning (RLGS) (Costello et al., 2000; Nat Genet.;24(2):132-8), methylation-sensitive representational difference analysis (MS-RDA) (Ushijima et al., Proc Natl Acad Sci USA. 1997 Mar. 18; 94(6):2284-9), and differential methylation hybridization (DMH) (Huang et al., Cancer Res. 1997 Mar. 15; 57(6):1030-4). Restriction endonucleases can be methylation dependent in their digestion activity. This specificity can be used to differentiate methylated and unmethylated sequences. Certain restriction enzymes, for example BstUl, Hpall and Notl are sensitive to methylated recognition sequences. Others, such as McrBC, are specific for methylated sequences.

As an example, differential methylation hybridisation (DMH) (Huang et al., as above]) requires an initial fragmentation of the genome with a bulk genome restriction enzyme, such as Msel, which fragments the genome into lengths of less than 200 bp. Following this step, the genome fragments are digested using a methylation-sensitive restriction endonuclease (MREs), or in some versions of the technique, a cocktail of MREs to improve coverage. Depending on the specificity of enzyme or enzymes used, either the methylated or the unmethylated sequences will be degraded. Digested sequences will not be amplified in a subsequent PCR step. The resultant PCR products are suitable for further processing and analysis by sequencing or microarray hybridisation in combination with fluorescent dyes.

Suitably, the present methods utilise a DNA methylation profile generating by a method comprising the use of one or more MREs.

Suitable comparators can be used to investigate methylation state between conditions. DNA from healthy subjects can be compared with aged or diseased subjects to detect changes in methylation state (Huang et al., Hum Mol Genet. 1999 March;8(3):459-70). Alternatively, a methylation-insensitive version of the secondary digest enzyme, such as the Hpall isoschizomer Mspl, can be used to generate a control sample, so that intra- or inter-genomic DNA methylation comparisons can be made (Khulan et al., Genome Res. 2006 August;16(8):1046-55).

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. Alternatively, the method can encompass amplifying intact DNA after restriction enzyme digestion, thereby only amplifying DNA that was not cleaved by the restriction enzyme in the area amplified. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

Suitably, the digestion of nucleic acid is detected by selective hybridization of a probe or primer to the undigested nucleic acid. Alternatively, the probe selectively hybridizes to both digested and undigested nucleic acid but facilitates differentiation between both forms, e.g., by electrophoresis. Suitable detection methods for achieving selective hybridization to a hybridization probe include, for example, Southern or other nucleic acid hybridization.

Suitable hybridization conditions may be determined based on the melting temperature (Tm) of a nucleic acid duplex comprising the probe. The skilled artisan will be aware that optimum hybridization reaction conditions should be determined empirically for each probe, although some generalities can be applied. Preferably, hybridizations employing short oligonucleotide probes are performed at low to medium stringency. In the case of a GC rich probe or primer or a longer probe or primer a high stringency hybridization and/or wash is preferred. A high stringency is defined herein as being a hybridization and/or wash carried out in about 0.1×SSC buffer and/or about 0.1% (w/v) SDS, or lower salt concentration, and/or at a temperature of at least 65° C., or equivalent conditions. Reference herein to a particular level of stringency encompasses equivalent conditions using wash/hybridization solutions other than SSC known to those skilled in the art.

### Affinity Enrichment Based Methods

Distinction of methylated from unmethylated DNA can be accomplished by the use of antibodies, such as anti-5mC, and/or methylated-CpG binding proteins, that contain a methyl-CpG-binding domain (MBD). The antibodies of MBD-domain proteins are able to specifically isolate methylated DNA over unmethylated DNA. Methods that utilize antibodies are commonly referred to as MeDIP, whilst methods utilizing methylated-CpG binding proteins are often known as MBD or MIRA approaches.

These methods require initial fragmentation of the genome, which can be carried out with bulk genome digest with an enzyme such as Msel, which cuts frequently, followed by affinity purification of methylated fragments. The input DNA can be compared to the purified methylated DNA by microarray hybridisation or sequencing to obtain comparative analysis of methylation levels at specific sites.

Further variants of affinity enrichment-based methods are available, such as MethylCap-Seq or MBD-Seq. These methods reduce sample complexity by using a salt gradient to elute methylated DNA fragments in a methy-CpG-abundance dependent manner, segregating CpG islands and other highly methylated loci from less CpG dense loci. The fractions can then be sequenced separately improving sequence coverage.

### Single Molecule Sequencing-Based Approaches

Contemporary sequencing methods are able to sequence single molecules directly. Single-molecule real-time (SMRT) DNA sequencing is available, for example the Sequel systems from Pacific Biosciences and has been shown to be able to identify modified bases such as methylated cytosine based on the polymerase kinetics. Nanopore sequencing devices, such as the MinION nanopore sequencer from Oxford Nanopore Technologies, which are able to individually sequence long strands of DNA, are also able to detect base modifications, including methylation.

### DNA Methylation Sites

Suitably, a DNA methylation site may refer to the presence or absence of a 5mC at a single cytosine, suitably a single CpG dinucleotide.

Suitably, a DNA methylation site may refer to the presence or absence of methylation (i.e. the number of 5mC or percentage of 5mC) across a plurality of CpG sites within a DNA region. Suitably, a DNA site methylation site may refer to the level of methylation (i.e. the number of 5mC or percentage of 5mC) across a plurality of CpG sites within a DNA region. A “DNA region” may refer to a specific section of genomic DNA. These DNA regions may be specified either by reference to a gene name or a set of chromosomal coordinates. Both the gene names and the chromosomal coordinates would be well known to, and understood by, the person of skill in the art.

Suitably, gene names and/or coordinates may be based on the “Tasha” dog reference genome (https://www.ncbi.nlm.nih.gov/assembly/GCF_000002285.5; Jagannathan et al.; Genes (Bsael); 2021; 12(6); 847).

The DNA region may define a section of DNA in proximity to the promoter of a gene, for example. Promoter regions are known to be rich in CpG. By way of example, the DNA region may refer to about 3kb upstream to about 3kb downstream; about 2kb upstream to about 2kb downstream; about 2kb upstream to about 1kb downstream; about 2kb upstream to about 0.5kb downstream; about 1kb upstream to about 0.5kb downstream; about 0.5kb upstream to about 0.5kb downstream of a promoter. Suitably, the DNA region may refer to about 1kb upstream to about 0.5kb downstream of a promoter.

Suitably, the DNA region may comprise or consist of CpG sites that are less than about 5000, less than about 4000, less than about 3000, less than about 2000, less than about 1000, less than about 500, or less than about 200 bases apart.

Suitably, the DNA region may comprise or consist of CpG sites that are between about 200 to about 5000, about 200 to about 4000, about 200 to about 3000, about 200 to about 2000, or about 200 to about 1000 bases apart.

Suitably, the DNA region may comprise one or more CpG islands. Suitably, the DNA region may consist of a CpG island.

A “CpG island” may refer to a DNA region comprising at least 200 bp, a GC percentage greater than 50%, and an observed-to-expected CpG ratio greater than 60%.

Suitably, the DNA methylation sites do not comprise X and/or Y chromosome CpGs.

Suitably, the DNA methylation sites do not comprise CpGs known to comprise a SNP at the CpG.

Reference to each of the genes/DNA regions detailed above should be understood as a reference to all forms of these molecules and to fragments or variants thereof. As would be appreciated by the person of skill in the art, some genes are known to exhibit allelic variation between individuals or single nucleotide polymorphisms. Variants include nucleic acid sequences from the same region sharing at least 90%, 95%, 98%, 99% sequence identity i.e. having one or more deletions, additions, substitutions, inverted sequences etc. relative to the DNA regions described herein. Accordingly, the present invention should be understood to extend to such variants which, in terms of the present applications, achieve the same outcome despite the fact that minor genetic variations between the actual nucleic acid sequences may exist between individuals. The present invention should therefore be understood to extend to all forms of DNA which arise from any other mutation, polymorphic or allelic variation.

In terms of screening for the methylation of these gene regions, it should be understood that the assays can be designed to screen for specific DNA. It is well within the skill of the person in the art to choose which strand to analyse and to target that strand based on the chromosomal coordinates. In some circumstances, assays may be established to screen both strands.

“Methylation status” may be understood as a reference to the presence, absence and/or quantity of methylation at a particular nucleotide, or nucleotides, within a DNA region. The methylation status of a particular DNA sequence (e.g. DNA region as described herein) can indicate the methylation state of every base in the sequence or can indicate the methylation state of a subset of the base pairs (e.g, of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.”

Suitably, DNA methylation may be determined using an EM-Seq strategy. In such methods, a methylation level can be determined as the fraction of ‘C’ bases out of ‘C’+‘U’ total bases at a target CpG site “i” following an enzyme and APOBEC3A conversion treatment. In other embodiments, the methylation level can be determined as the fraction of ‘C’ bases out of ‘C’+‘T’ total bases at site “i” following enzyme and APOBEC3A conversion treatment and subsequent nucleic acid amplification. The mean methylation level at each site may then be evaluated to determine if one or more threshold is met.

In some embodiments, in particular when bisulfite conversion and sequencing methods are used, a methylation level can be determined as the fraction of ‘C’ bases out of ‘C’+‘U’ total bases at a target CpG site “i” following a bisulfite treatment. In other embodiments, the methylation level can be determined as the fraction of ‘C’ bases out of ‘C’+‘T’ total bases at site “i” following a bisulfite treatment and subsequent nucleic acid amplification. The mean methylation level at each site may then be evaluated to determine if one or more threshold is met.

Alternatively, a methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of the methylation status. This is of particular use when it is desirable to compare the methylation status of a sequence in a sample to a threshold value.

The present invention is not to be limited by a precise number of methylated residues that are considered to indicative of biological age, because some variation between samples will occur. The present invention is also not necessarily limited by positioning of the methylated residue (e.g. a specific methylation site).

In one embodiment, a screening method can be employed which is specifically directed to assessing the methylation status of one or more specific cytosine residues or the corresponding cytosine at position n+1 on the opposite DNA strand.

### Enrichment and Detection Methods

Determining a DNA methylation profile may comprise a step of enriching a DNA sample for selected DNA regions. For example, the methods may comprise a step of enriching a DNA sample for DNA regions comprising the DNA methylation sites which comprise the DNA methylation profile.

Suitable enrichment methods are known in the art and include, for example, amplification or hybridisation based methods. Amplification enrichment typically refers to e.g. PCR based enrichment using primers against the DNA regions to be enriched. Any suitable amplification format may be used, such as, for example, polymerase chain reaction (PCR), rolling circle amplification (RCA), inverse polymerase chain reaction (iPCR), in situ PCR, strand displacement amplification, or cycling probe technology.

Hybridisation enrichment or capture-based enrichment typically refers to the use of hybridisation probes (or capture probes) that hybridise to DNA regions to be enriched.

The hybridisation probe(s) may be attached directly to a solid support, or may comprise a moiety, e.g. biotin, to allow binding to a solid support suitable for capturing biotin moieties (e.g. beads coated with streptavidin). In any case, DNA comprising sequence which is complementary to the probe may be captured thus allowing to separate DNA comprising DNA regions of interest from not comprising the DNA regions of interest. Hence, such a capturing steps allows to enrich for the DNA regions of interest. For example, the DNA regions may be DNA regions in proximity to gene promoters.

An array used herein can vary depending on the probe composition and desired use of the array. For example, the nucleic acids (or CpG sites) detected in an array can be at least 10, 100, 1,000, 10,000, 0.1 million, 1 million, 10 million, 100 million or more. Alternatively or additionally, the nucleic acids (or CpG sites) detected can be selected to be no more than 100 million, 10 million, 1 million, 0.1 million, 10,000, 1,000, 100 or less. Similar ranges can be achieved using nucleic acid sequencing approaches such as those known in the art; e.g. Next Generation or massively parallel sequencing.

Suitably, an enrichment step may be performed before or after the step of separating or differentially treating methylated and unmethylated DNA.

As used herein, the term “enriching” or “enrichment” for “DNA” or “DNA regions” means a process by which the (absolute) amount and/or proportion of the DNA comprising the desired sequence(s) is increased compared to the amount and/or proportion of DNA comprising the desired sequence(s) in the starting material. In this regard, enrichment by amplification increases the amount and proportion of the desired sequence(s). Enrichment by capture-based enrichment increases the proportion of DNA comprising the desired sequence(s).

Following processing of the DNA to distinguish methylated and unmethylated sites, the present methods may further comprise the step of identifying the sites which were methylated or unmethylated (i.e. in the original sample).

The identification step may comprise any suitable method known in the art, for example array detection or sequencing (e.g. next generation sequencing).

A sequencing identification step preferably comprises next generation sequencing (massively parallel or high throughput sequencing). Next generation sequencing methods are well known in the art, and in principle, any method may be contemplated to be used in the invention. Next generation sequencing technologies may be performed according to the manufacturer's instructions (as e.g. provided by Roche, Illumina or Applied Biosystems).

In a preferred embodiment, the sample is treated by converting DNA methylation using enzymatic reactions, performing whole genome library preparation and measuring the methylation profile by sequencing (EM-Seq).

In a particularly preferred embodiment, the sample is treated by converting DNA methylation using enzymatic reactions, performing whole genome library preparation, hybridizing the whole-genome-converted library preparation to capture probes (preferably capture probes capable of capturing DNA regions in proximity to gene promoters); and measuring the methylation profile by sequencing (EM-Seq).

### DNA Methylation Profile

A “DNA methylation profile” or “methylation profile” may refer to the presence, absence, quantity or level of 5mC at one or more DNA methylation sites. Preferably, “methylation profile” refers to the presence, absence, quantity or level of 5mC at a plurality of DNA methylation sites. Thus, the presence, absence, quantity or level of 5mC at each individual DNA methylation site within the plurality of sites may be assessed and contribute to the determination of the biological age of the dog. The quality and/or the power of the methods may thus be improved by combining values from multiple DNA methylation markers.

Suitably, the present biological clock comprises the methylation profile from a plurality of methylation sites.

Suitably, presence or absence of 5mC from at least 3, at least 5, at least 10, at least 20, at least 50, at least 100, at least 200, at least 500, at least 1000, at least 2000, at least 5000, at least 10000, at least 50000, at least 10000, at least 250000, or at least 500000 DNA methylation sites may be used to determine the biological age of the dog.

Suitably, the methylation profile may refer to the presence or absence of 5mC from at least 100, at least 200, at least 500, at least 1000 or at least 2000 DNA methylation sites.

Suitably, the methylation profile may refer to the presence or absence of 5mC from about 100, about 200, about 500, about 1000 or about 2000 DNA methylation sites.

In order to generate a biological clock, an initial methylation profile may be processed or streamlined to produce a restricted methylation profile which is then used to generate the biological clock.

By way of example, an initial methylation profile may be processed or streamlined by—for example—using DNA regions rather than individual cytosines, by selecting a subset of methylation sites that are associated with a particular physiological or biochemical pathway, performing a correlation analysis and retaining one or more representative DNA methylation sites per cluster, or performing differential analysis to pre-select DNA methylation sites or retain DNA methylation sites that vary more between young and old dogs,

For example, the DNA region(s) may be any DNA region(s) as defined herein.

Suitably, the methylation profile may refer to DNA methylation sites of genes that are associated with a particular physiological or biochemical pathway. As such, the methylation profile may enable a biological age of a particular tissue, organ, or physiological system to be determined. Determining a biological age for a particular tissue, organ or physiological system may advantageously allow the method to be utilised in a way which focuses on pathologies and diseases of that tissue, organ or physiological system. For example, if a particular breed of dog is known to be associated with muscular or cardiovascular disease, it may be advantageous to determine a biological age for that physiological system.

Suitably, the physiological system may be the immune, gastrointestinal, urinary, muscular, cardiovascular, and/or neurological system.

A biological age for a particular tissue, organ, or physiological system may be determined using a DNA methylation profile comprising, or consisting of, methylation sites from genes that are preferentially or specifically expressed by that tissue, organ, or physiological system. Classifications of genes by a particular tissue, organ, or physiological system are publicly available at, for example, Gene Ontology (http://geneontology.org/), the KEGG pathway database (https://www.genome.jp/kegg/), or MSIgDB (https://www.gsea-msigdb.org/gsea/msigdb/index.jsp).

In some embodiments, a threshold selects those sites having the highest-ranked mean methylation values for epigenetic age predictors. For example, the threshold can be those sites having a mean methylation level that is the top 50%, the top 40%, the top 30%, the top 20%, the top 10%, the top 5%, the top 4%, the top 3%, the top 2%, or the top 1% of mean methylation levels across all sites “i” tested for a predictor, e.g., a biological clock.

Alternatively, the threshold can be those sites having a mean methylation level that is at a percentile rank greater than or equivalent to 50, 60, 70, 80, 90, 95, 96, 97, 98, or 99. In other embodiments, a threshold can be based on the absolute value of the mean methylation level. For instance, the threshold can be those sites having a mean methylation level that is greater than 99%, greater than 98%, greater than 97%, greater than 96%, greater than 95%, greater than 90%, greater than 80%, greater than 70%, greater than 60%, greater than 50%, greater than 40%, greater than 30%, greater than 20%, greater than 10%, greater than 9%, greater than 8%, greater than 7%, greater than 6%, greater than 5%, greater than 4%, greater than 3%, or greater than 2%. The relative and absolute thresholds can be applied to the mean methylation level at each site “i” individually or in combination. As an illustration of a combined threshold application, one may select a subset of sites that are in the top 3% of all sites tested by mean methylation level and also have an absolute mean methylation level of greater than 6%. The result of this selection process is a DNA methylation profile, of specific hypermethylated sites (e.g., CpG sites) that are considered the most informative for biological age determination.

Suitably, the DNA methylation profile may comprise at least one methylation site as listed in Table 1.

Suitably, the methylation site(s) may be defined as the methylation markers present in any one or more of SEQ ID NO: 1-255. SEQ ID NO: 1-255 show the sequence either side of the methylation marker in the “Tasha” dog reference genome (https://www.ncbi.nlm.nih.gov/assembly/GCF_000002285.5; Jagannathan et al.; Genes (Bsael); 2021; 12(6); 847). The “CG” methylation marker are the 26^{th} and 27^{th} nucleotides in the sequence (i.e. there are 25 nucleotides preceding the methylation marker and 25 nucleotides following the methylation marker).

Suitably, the methylation site may be defined as the intervening position in the column labelled “Site” in Table 1. For example, for site chr1:32205238-32205240, the methylation marker is chr1: 32205239.Suitably, the DNA methylation profile may comprise at least 3, at least 5, at least 10, at least 20, at least 50, at least 100, at least 150, at least 200 or preferably each of the methylation sites as listed in Table 1.

Suitably, the DNA methylation profile may comprise the methylation sites chr2.32494387.32494389; chr6.45773846.45773848; and chr16.8886545.8886547. These sites are shown in Table 2.

Suitably, the DNA methylation profile may comprise the methylation sites chr2.32494387.32494389; chr6.45773846.45773848; chr16.8886545.8886547 chr5.61645225.61645227; and chr15.10856498.10856500. These sites are shown in Table 3.

Suitably, the DNA methylation profile may comprise the methylation sites chr2.32494387.32494389; chr6.45773846.45773848; chr16.8886545.8886547 chr5.61645225.61645227; chr15.10856498.10856500; chr33.26512711.26512713; chr20.57347921.57347923; chr12.12684190.12684192; chr24.30860619.30860621; and chr33.26512692.26512694. These sites are shown in Table 4.

Suitably, the DNA methylation profile may comprise the methylation sites chr2.32494387.32494389; chr6.45773846.45773848; chr16.8886545.8886547 chr5.61645225.61645227; chr15.10856498.10856500; chr33.26512711.26512713; chr20.57347921.57347923; chr12.12684190.12684192; chr24.30860619.30860621; chr33.26512692.26512694; chr10.7411293.7411295; chr1.22213697.22213699; chr5.32711703.32711705; chr24.21051024.21051026; chr5.21480151.21480153; chr17.33851462.33851464; chr3.46843750.46843752; chr13.37474592.37474594; chr33.19810904.19810906; and chr20.44942213.44942215. These sites are shown in Table 5.

Suitably, the DNA methylation profile may comprise the methylation sites chr2.32494387.32494389; chr6.45773846.45773848; chr16.8886545.8886547 chr5.61645225.61645227; chr15.10856498.10856500; chr33.26512711.26512713; chr20.57347921.57347923; chr12.12684190.12684192; chr24.30860619.30860621; chr33.26512692.26512694; chr10.7411293.7411295; chr1.22213697.22213699; chr5.32711703.32711705; chr24.21051024.21051026; chr5.21480151.21480153; chr17.33851462.33851464; chr3.46843750.46843752; chr13.37474592.37474594; chr33.19810904.19810906; chr20.44942213.44942215; chr20.54301453.54301455; chr35.23852932.23852934; chr6.60675998.60676000; chr22.46374563.46374565; chr24.12518910.12518912; chr16.8886356.8886358; chr21.43490796.43490798; chr11.53576970.53576972; chr4.36180013.36180015; chr4.43217432.43217434; chr9.35985983.35985985; chr32.40409729.40409731; chr23.37987526.37987528; chr18.44679236.44679238; chr21.16233628.16233630; chr10.18237423.18237425; chr30.8031060.8031062; chr1.121182146.121182148; chr9.50976682.50976684; chr14.58424689.58424691; chr20.49940551.49940553; chr2.31655107.31655109; chr18.46990963.46990965; chr20.46098568.46098570; chr10.68649436.68649438; chr9.25415871.25415873; chr14.39735273.39735275; chr11.33523800.33523802; chr5.32347978.32347980; and chr19.44679975.44679977.

### Determination of DNA Methylation Sites/DNA Methylation Profiles Indicative of Biological Age

The present invention comprises utilising a DNA methylation profile to determine a biological age of a dog. As such, the present invention comprises utilising a DNA methylation profile to generate a biological clock. The present biological clock may also be referred to as an ‘epigenetic clock’.

The provision of DNA methylation sites or a DNA methylation profile that is indicative of biological age may be achieved through training datasets and machine learning approaches, for example. Suitably, the machine learning approaches may be supervised machine learning approaches.

By way of example, DNA methylation sites or a DNA methylation profile may be trained against a dataset comprising dogs of a known chronological age. Suitably, the DNA methylation sites or a DNA methylation profile may be trained against a dataset comprising dogs of a known chronological age in combination with known breed and/or sex.

For example, models for DNA methylation sites or a DNA methylation profile indicative of biological age may be provided by training a dataset of methylation status at a plurality of DNA methylation sites against a training dataset of dogs with a known chronological age using a machine learning framework, and testing against a with-held cohort to validate the veracity of the model.

The machine learning framework may comprise fitting a penalised regression to a training dataset of dogs with a known chronological age (and optionally breed and/or sex); for example using glmnet R package.

The machine learning framework may comprise fitting an elastic net regression to a training dataset of dogs with a known chronological age (and optionally breed and/or sex); for example using glmnet R package.

Suitably, the machine learning framework may comprise fitting a penalised regression, such as an elastic net regression, of chronological age explained by a DNA methylation profile, (and optionally breed, age and/or sex).

Suitably, the machine learning framework may comprise fitting a penalised regression, such as an elastic net regression, of chronological age explained by a DNA methylation profile, breed, age and sex.

Suitably, the machine learning framework may be used to determine a model comprising a set of DNA methylation sites or a DNA methylation profile that is indicative of biological age.

The model may comprise the methylation status at a plurality of DNA methylation sites; wherein the methylation status at each site is considered in the model by multiplying by a coefficient value.

The coefficient value for each parameter typically depends on the measurement units of all the variables in the model. As would be understood by the skilled person, the value for each coefficient value will therefore depend on, for example, the number and nature of the different parameters used in the model and the nature of the training data provided. Accordingly, routine statistical methods may be applied to a training data set in order to arrive at coefficient values.

Suitably, sex may be coded as a numerical value with 0 for female and 1 for male.

Suitably, breed may be coded as a numerical value with 0 for small breeds and 1 for medium breeds.

Suitably, the machine learning platform may comprise one or more deep neural networks. Neural Networks are collections of neurons (also called units) connected in an acyclic graph. Neural Network models are often organized into distinct layers of neurons. For most neural networks, the most common layer type is the fully-connected layer in which neurons between two adjacent layers are fully pairwise connected, but neurons within a single layer share no connections. One of the main features of deep neural networks is that neurons are controlled by non-linear activation functions. This non-linearity combined with the deep architecture make possible more complex combinations of the input features leading ultimately to a wider understanding of the relationships between them and as a result to a more reliable final output. Deep neural networks have been applied for many types of data ranging from structural data to chemical descriptors or transcriptomics data.

Suitably, the machine learning platform comprises one or generative adversarial networks. Suitably, the machine learning platform comprises an adversarial autoencoder architecture. Suitably, the machine learning platform comprises a feature importance analysis for ranking DNA methylation site by their importance in biological age determination.

The biological age of the dog may be expressed in terms of years, months, days, etc.

### Comparison to a Reference or Control

The present method may further comprise a step of comparing the difference in DNA methylation at one or more sites in the test sample to one or more reference or controls. The presence or absence of DNA methylation at one or more sites in the reference or control may be associated with a pre-defined biological age. In some embodiments, the reference value is a value obtained previously for a subject or group of subjects with a known biological age. The reference value may be based on a known DNA methylation status at one or more sites, e.g. a mean or median level, from a group of subjects with known chronological age, breed, and/or sex.

Combining the Biomarker Levels with Further Measures and/or Characteristics

Suitably, the present method further comprises explaining chronological age by DNA methylation profile in combination with one or more of the breed and/or sex of the dog.

### Subject Stratification

The biological age determined by the method of the present invention may also be compared to one or more pre-determined thresholds (i.e. difference to chronological age). Using such thresholds, subjects may be stratified into categories which are indicative of determined risk, e.g. low, medium or high determined risk. The extent of the divergence from the thresholds is useful to determine which subjects would benefit most from certain interventions. In this way, dietary intervention and modification of lifestyle can be optimised.

### Method for Selecting/Monitoring a Lifestyle Regime, Dietary Regime or Therapeutic Intervention of a Subject

In a further aspect, the present invention provides a method for selecting a lifestyle regime, dietary regime or therapeutic intervention for a subject. The modification in lifestyle may be any change as described herein, e.g. a dietary intervention and/or a change in exercise regime. The modification in lifestyle may be administration of a therapeutic modality.

The lifestyle regime, dietary regime or therapeutic intervention may be applied to the dog for any suitable period of time. After said period of time, the dog's biological age may be determined again using the present method in order to determine the efficacy of the lifestyle regime, dietary regime or therapeutic intervention for improving the biological age of the dog. By way of example, the lifestyle regime, dietary regime or therapeutic intervention may be applied for at least 2, at least 4, at least 8, at least 16, at least 32, or at least 64 weeks. The lifestyle regime, dietary regime or therapeutic intervention may be applied for at least 3, at least 6, at least 12, at least 24, at least 36, at least 48 or at least 60 months.

The lifestyle regime, dietary regime or therapeutic intervention may be referred to as an anti-aging lifestyle regime, dietary regime or therapeutic intervention.

Preferably the modification is a dietary intervention as described herein. By the term “dietary intervention” it is meant an external factor applied to a subject which causes a change in the subject's diet. More preferably the dietary intervention includes the administration of at least dietary product or dietary regimen or a nutritional supplement.

The dietary regime may be a meal, a regime of meals, a supplement or a regime of supplements, or combinations of a meal and a supplement, or combinations of a meal and multiple supplements.

The dietary intervention or dietary product described herein may be any suitable dietary regime, for example, a calorie-restricted diet, a senior diet, a low protein diet, a phosphorous diet, low protein diet, potassium supplement diet, polyunsaturated fatty acids (PUFA) supplement diet, anti-oxidant supplement diet, a vitamin B supplement diet, liquid diet, selenium supplement diet, omega 3-6 ratio diet, or diets supplemented with carnitine, branched chain amino acids or derivatives, nucleotides, nicotinamide precursors such as nicotinamide mononucleotide (MNM) or nicotinamide riboside (NR) or any combination of the above.

Suitably, the dietary intervention or dietary product may be a calorie-restricted diet, a senior diet, or a low protein diet. Suitably, the dietary intervention or dietary product may be a calorie-restricted diet. Suitably, the dietary intervention or dietary product may be a low protein diet.

A dietary intervention may be determined based on the baseline maintenance energy requirement (MER) of the dog. Suitably, the MER may be the amount of food that stabilizes the dog's body weight (less than 5% change over three weeks).

By way of example, it is generally understood that younger, growing dogs benefit from a high energy/high protein diet; however, older dogs may have a lower energy requirement and therefore diets can be appropriately modified. In particular, many manufacturers produce a ‘senior’ range of dog food which is lower in calories, higher in fibre but has suitable levels of protein and fat for an older dog.

Suitably, a calorie-restricted diet may comprise about 50%, about 55%, about 60%, about 65%, about 75%, about 80%, about 85%, or about 90% of the dog's MER. Suitably, a calorie-restricted diet may comprise about 60% or about 75% of the dog's MER.

Suitably, a low-protein diet may comprise less than 20% protein (% dry matter). For example, a low-protein diet may comprise less than 19% (% dry matter).

These diets are generally recommended based upon the chronological age of a dog. For example, it may be recommended that a dog is switched to a senior diet around 7 or 8 years old. However, in the context of the present invention, the determination of an increased mortality risk for a dog compared to what would be expected given its chronological age may allow a determination to switch the dog to a senior diet at an earlier age. In contrast, a dog with a reduced mortality risk compared to its chronological age may be able to stay on an adult diet for longer.

The dietary intervention may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts (or extracts thereof). Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

Modifying a lifestyle of the subject also includes indicating a need for the subject to change lifestyle, e.g. prescribing more exercise. Similar to a dietary intervention, the determination of an increased mortality risk for a dog compared to what would be expected given its chronological age may allow a determination a switch the dog to an appropriate exercise regime.

Modifying a lifestyle of the subject also includes recommending a therapeutic modality or regimen. The therapeutic modality or regimen may be a modality useful in treating and/or preventing—for example—arthritis, dental diseases, endocrine disorders, heart disease, diabetes, liver disease, kidney disease, prostate disorders, cancer and behavioural or cognitive disorders. Suitably, prophylactic therapies may be administered to a dog identified as being at risk of such disorders due to increased biological age. In other embodiments, dogs determined to be at risk of certain conditions due to increased biological age may be monitored more regularly so that diagnosis and treatment can begin as early as possible.

The present invention is also directed to monitoring and/or determining the efficacy of an anti-ageing therapy or developing an anti-ageing therapy. The anti-aging therapy may comprise, for example, a “rejuvenation” intervention. A rejuvenation intervention aims to cause a reduction in the epigenetic or biological age of the subject. Suitably, the rejuvenation intervention may reprogram epigenetic age to that of a very young dog. Examples of such rejuvenation interventions include, but are not limited to, a gene therapy that reprograms epigenetic age, suitably to that of a very young dog. The present methods to monitor and/or determine the efficacy of a lifestyle regime, dietary regime or therapeutic intervention or develop a lifestyle regime, dietary regime or therapeutic intervention to reduce biological age are particularly applicable to this aspect.

The present invention may thus advantageously enable the identification of dogs that are expected to respond particularly well to a given intervention (e.g. lifestyle regime, dietary regime or therapeutic intervention). The intervention can thus be applied in a more targeted manner to dogs that are expected to respond.

In one aspect, the present invention provides a method for determining the efficacy of a lifestyle regime, dietary regime or therapeutic intervention for improving the biological age of a dog, said method comprising:
a) applying a lifestyle regime, dietary regime or therapeutic intervention to the dog, wherein the lifestyle regime, dietary regime or therapeutic intervention has been selected according to the first aspect of the invention;
b) after a time period of applying the lifestyle regime, dietary regime or therapeutic intervention to the dog; determining a biological age of the dog using a DNA methylation profile from a sample obtained from the dog;
c) determining if there has been a change in the biological age of the dog after the time period of following the lifestyle regime, dietary regime or therapeutic intervention.

In another aspect, the invention relates to a method for determining the efficacy of a lifestyle regime, dietary regime or therapeutic intervention for improving the biological age of a dog, said method comprising:
a) determining a biological age of the dog using a DNA methylation profile from a sample obtained from the dog;
b) applying a lifestyle regime, dietary regime or therapeutic intervention selected based on the biological age determined in step a) to the dog;
c) after a time period of applying the lifestyle regime, dietary regime or therapeutic intervention to the dog; determining a biological age of the dog using a DNA methylation profile from a sample obtained from the dog;
d) determining if there has been a change in the biological age of the dog between step a) and step c).

In a further aspect, the invention relates to a method for determining the efficacy of a lifestyle regime, dietary regime or therapeutic intervention for improving the biological age of a dog, said method comprising:
a) determining a first biological age of the dog using a DNA methylation profile from a sample obtained from the dog;
b) applying a lifestyle regime, dietary regime or therapeutic intervention to the dog;
c) after a time period of applying the lifestyle regime, dietary regime or therapeutic intervention to the dog; determining a second biological age of the dog using a DNA methylation profile from a second sample obtained from the dog;
d) determining if there has been a change in the first and second biological age of the dog after the time period of following the lifestyle regime, dietary regime or therapeutic intervention.

Suitably, the lifestyle regime, dietary regime or therapeutic intervention may have been applied to the dog for a period before the first biological age is determined; however, the effectiveness of the lifestyle regime, dietary regime or therapeutic intervention for improving the biological age of the dog (e.g. reducing the biological age, reducing an increase, or a rate of increase, in biological age) may still be monitored by determining a biological age at two or more times during the application of the lifestyle regime, dietary regime or therapeutic intervention.

Suitably, the present methods may comprise an ‘ecosystem’; in particular a digital ecosystem. Suitably, the present methods may comprise providing a sample obtained from the dog, optionally using a kit according to present invention; and (b) providing the sample (e.g. by mailing) for subsequent DNA extraction for the measurement of DNA methylation in the extracted DNA from the sample to obtain a DNA methylation profile.

The DNA methylation profile may then be used according to any of the present methods; preferably using a computer system or a computer program product according to the present invention.

The computer system or computer program may then prepare and share a report detailing the outcome of analysis/method in the form of e.g. selecting or recommending a suitable lifestyle regime, dietary regime or therapeutic intervention for a dog or any other outcome of the present methods.

Suitably, the sample may be a sample that can be obtained at home by a dog owner (e.g. not requiring a veterinarian or health-care professionals). Suitably, the sample may be a hair follicle, buccal swab or saliva sample.

### Use of a Dietary Intervention

In one aspect, the present invention provides a dietary intervention for use in improving the biological age of a dog, wherein the dietary intervention is administered to a dog with a biological age determined by the present method.

In another aspect, the present invention provides the use of a dietary intervention to improve the biological age of a dog, wherein the dietary intervention is administered to a dog with a biological age determined by the present method.

As described herein, the dietary intervention may be a dietary product or dietary regimen or a nutritional supplement.

### Computer Program Product

The present methods may be performed using a computer. Accordingly, the present methods may be performed in silico.

Suitably, the computer may prepare and share a report detailing the outcome of the present methods.

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to determine the biological age of a dog as described herein.

In another aspect, the present invention provides a computer program product comprising computer implementable instructions for causing a device to determine a biological age of the dog using a DNA methylation profile from the dog; and optionally select a suitable lifestyle regime, dietary regime or therapeutic intervention for the dog based on the biological age determined using the DNA methylation profile. The computer program product may also be given additional parameters or characteristics for the dog. As described herein, the additional parameters or characteristics may include chronological age, breed and sex.

In one embodiment, the user inputs into the device levels of one or more of DNA methylation markers as defined herein, optionally along with chronological age, breed and sex. The device then processes this information and provides a determination of a biological age for the dog. Alternatively, the device then processes this information and provides a determination of a suitable lifestyle regime, dietary regime or therapeutic intervention for the dog based on the biological age.

The device may generally be a server on a network. However, any device may be used as long as it can process biomarker data and/or additional parameters or characteristic data using a processor, a central processing unit (CPU) or the like. The device may, for example, be a smartphone, a tablet terminal or a personal computer and output information indicating the determined biological age for the dog or a determination of a suitable lifestyle regime, dietary regime or therapeutic intervention for the dog based on the biological age.

Those skilled in the art will understand that they can freely combine all features of the present invention described herein, without departing from the scope of the invention as disclosed.

### EXAMPLES

The invention will now be further described by way of examples, which are meant to serve to assist the skilled person in carrying out the invention and are not intended in any way to limit the scope of the invention.

### Example 1—Illustrative Method for Generating an Epigenetic Biological Clock

### Identification of DNA Methylation Sites

Whole blood samples from a canine cohort were analysed by performing DNA extraction, converting DNA methylation by using enzymatic reactions, performing whole genome library preparation, hybridizing the whole-genome-converted library preparation to capture probes directed against gene promoters and measuring the methylation profile by sequencing (EM-Seq).

The capture probes were directed against approximately 40,000 targets (promotor regions—approximately 1kb upstream and 0.5 downstream the promoter). These target regions comprise potential methylation sites of interest (individual cytosine residues that may be methylated).

The following bioinformatics steps were performed after sequencing and before further analysis:
Quality check of fastq using fastQC—https://www.bioinformatics.babraham.ac.uk/projects/fastqc/
Adapter trimming using trimGalore—https://www.bioinformatics.babraham.ac.uk/projects/trim_galore/
Align to dog genome using bwa-meth (https://github.com/brentp/bwa-meth) (or Bismark)—https://www.bioinformatics.babraham.ac.uk/projects/bismark/
Mark Duplicates using Picard—https://gatk.broadinstitute.org/hc/en-us/articles/360037052812-MarkDuplicates-Picard-
Call methylation using Methyldackel—https://github.com/dpryan79/MethylDackel

### Approaches for Filtering Sites to Generate a Restricted DNA Methylation Profile for Training the Biological Clock

Following determination of the methylation status of methylation sites in each sample, the initial methylation profile may be filtered/processed in order to generate a restricted methylation profile comprising fewer discrete methylation sites. The goal of this filtering is to provide a restricted methylation profile comprising e.g. between 50000 and 500000 methylation sites that can be used to train the biological clock.

Methods for filtering the initial methylation profile include:
1) Removing sites that are (un)methylated in all samples
2) Removing sites that do not have at least 5 counts in at least 90% of the samples
3) Removing chromosome X and/or Y sites

Further potential filtering steps to reduce the number of discrete methylation sites include:
1) Defining methylation sites as DNA regions (CpG islands, sites that are less than e.g. 1000 bp apart are considered as the same)
2) Restrict targets to e.g. inflammatory sites, for example genes associated with inflammation from GO/KEGG pathway and select the sites on these genes
3) Correlation analysis and retention of a representant(s) per cluster (e.g. Weighted correlation network analysis (WGCNA) (Langfelder & Horvath; BMC Bioinformatics; 9(559); 2008 or EBModules (Zollinger et al.; Biostatistics, 19(2), 153-168; (2018)).
4) Differential methylation analysis to pre-select DNA methylation sites (e.g. using logistic regression) or identify sites that vary more between young and old dogs (e.g. using variance analysis). Normalization (quantile) is performed before the filtering step if differential methylation analysis is performed (using function preprocessQuantile in R).

### Generating a Biological Clock

The dataset including information on the chronological age, breed and sex of a cohort of dogs is split into training and testing sets (e.g. ⅔ data for training and ⅓ for testing, ensuring a good split with regards to metadata; for example similar proportion of each breed/each sex in the training and in the testing set).

A penalized regression is fit between chronological age and the restricted DNA methylation profile, sex and breed in order to identify DNA methylation sites that are relevant for biological age (i.e. to generate a DNA methylation biological clock).

The penalization parameter is selected to provide a reasonable number of DNA methylation sites (e.g. max 1000) with a good model fit for biological age.

The model for biological age based on DNA methylation sites is then assessed on the testing set.

In the present example, the following steps were performed:
1) All methylation sites with less than 15 counts coverage were set as “missing”;
2) Imputation was performed using the “Boostme” algorithm (Zou et al.; BMC Genomics 19, 390 (2018)) and the training, testing and validation sets size was set to 200,000 randomly chosen sites;
3) ChrX sites were removed;
4) Site filtering was performed using EBmodule (Zollinger et al.; Biostatistics, 19(2), 153-168; (2018)); the dataset was separated into blocks of about 5000 sites by grouping the sites per target (1500 bp around the TSS) and per chromosome. A correlation matrix was then calculated on which EB modules as described in were applied—as described by Zollinger et al. Each module was then represented by the medoid site. Some sites do not belong to any modules and are called scattered sites (defined as described in Zollinger et al.);
5) Sites present on the mammalian CpG array were removed (Arneson et al.; Nature Communications; 13; 783; 2022);
6) Elastic net regression was adjusted on the 500K sites using chronological age (age at DNA collection) as the response variable, this selects 255 sites as forming the biological clock (see Table 1).

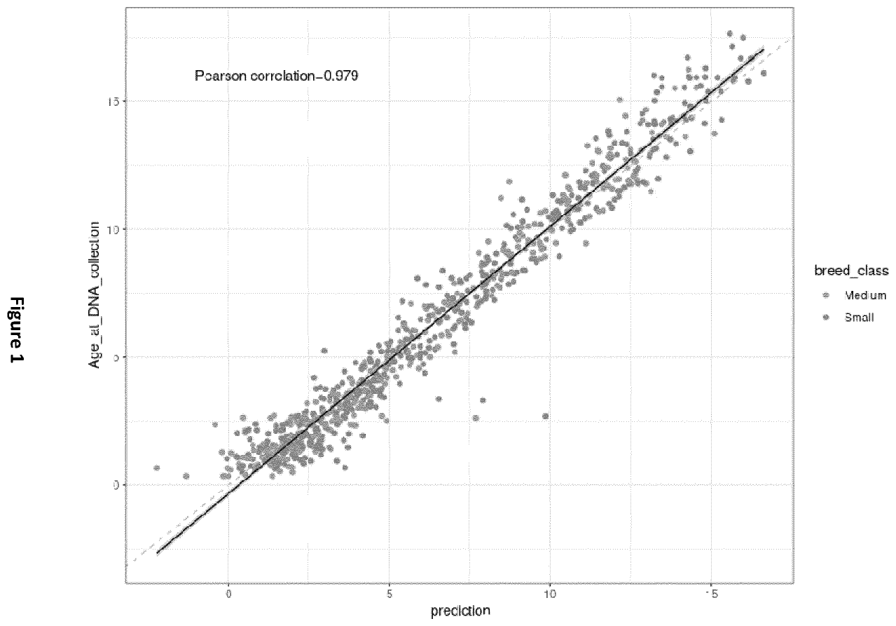

Biological age predicted from using the biological clock is highly correlated with chronological age (see FIG. 1).

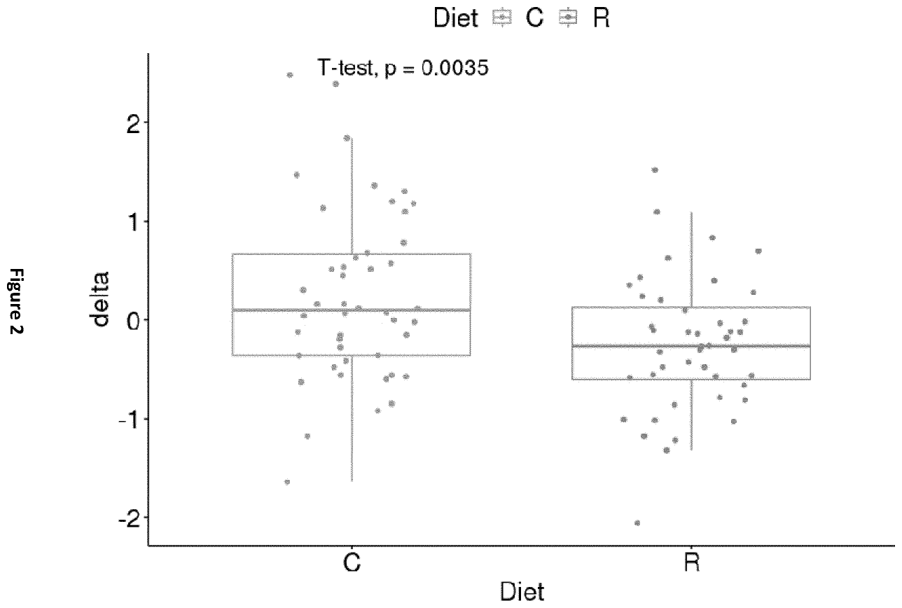

The biological clock was also validated using a calorie restriction study (see FIG. 2). Delta corresponds to the residuals of the regression model of Chronological Age vs predicted biological Age using the biological clock. Dogs on a calorie restriction were determined to have a significantly lower biological age (lower delta) compared to dogs on a control diet (FIG. 2).

A univariate analysis was performed to determine if each of the 255 sites alone was statistically significant in respect of associated with biological age (see Table 1).

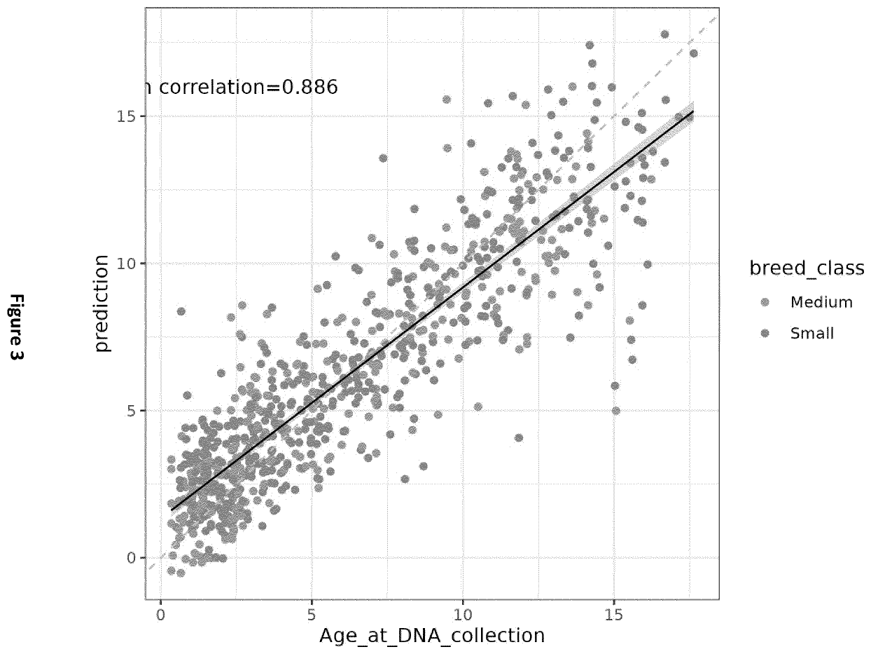

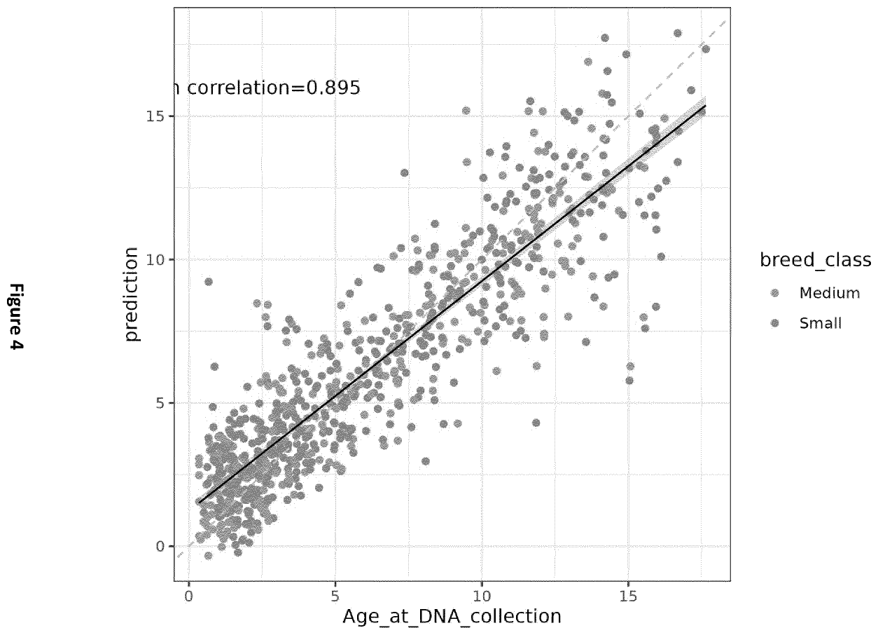

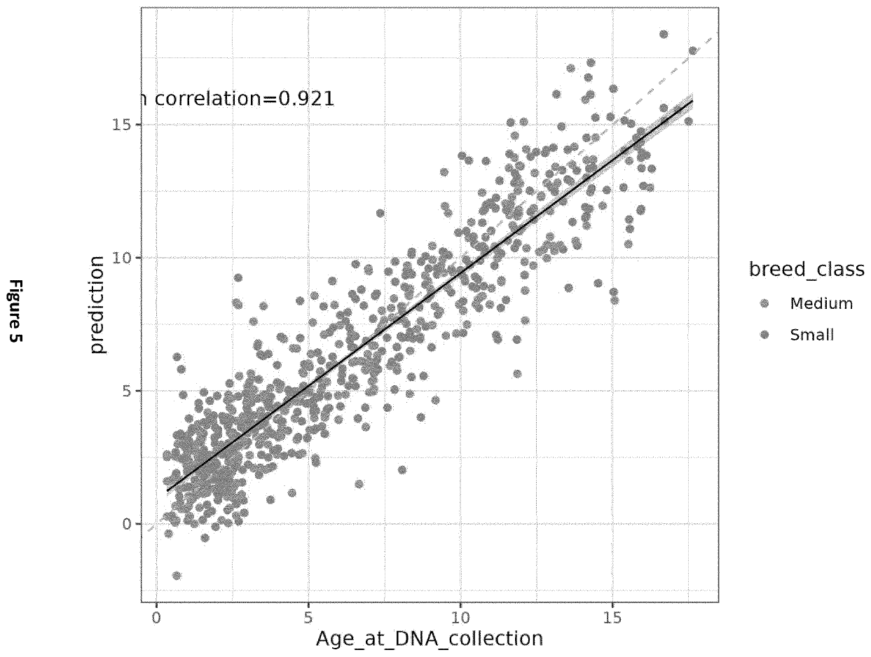

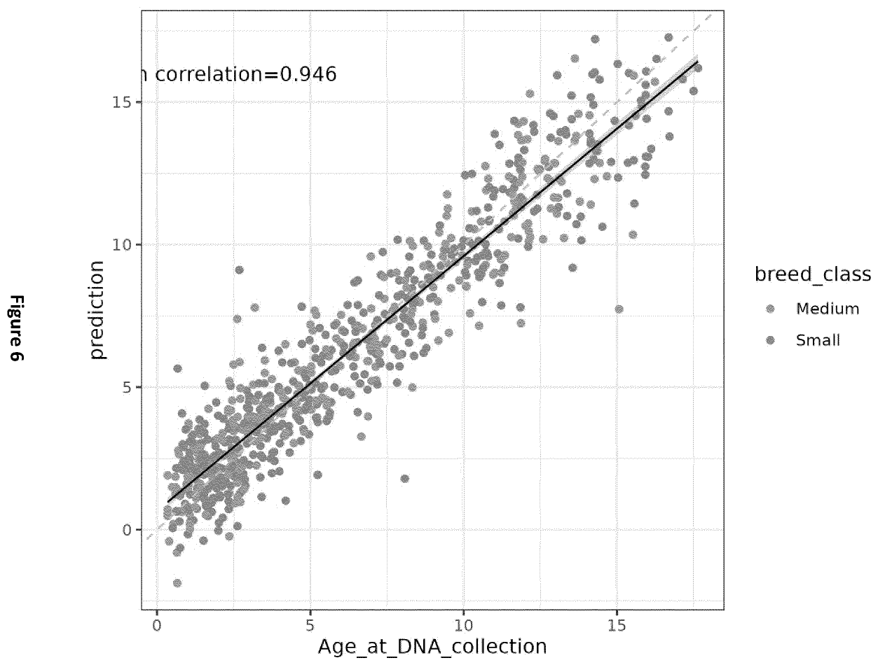

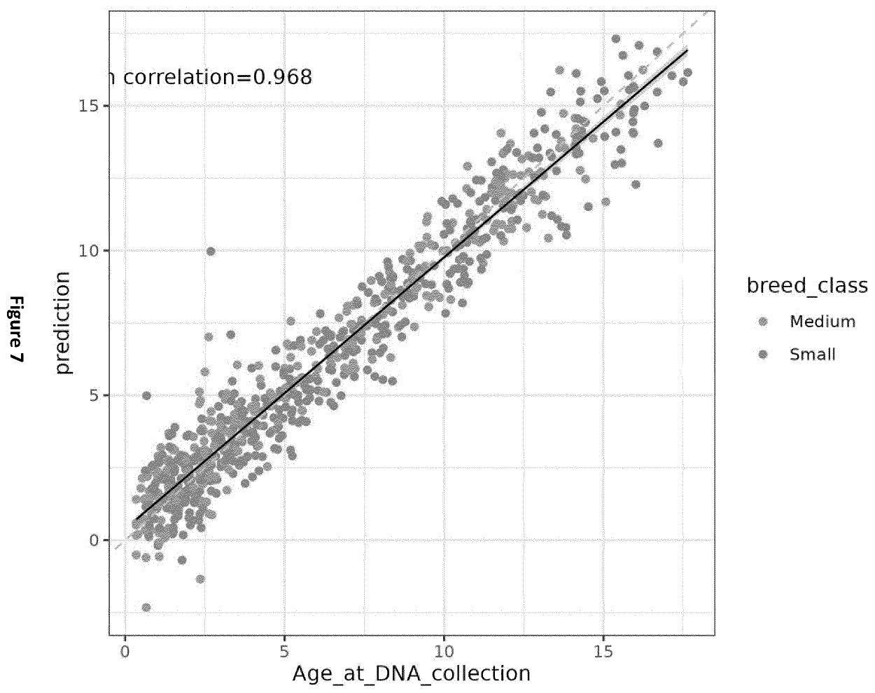

Further biological clocks were also generated using only the top 3, top 5, top 10, top 20 and top 50 sites from the complete list of sites shown in Table 1; and each was shown to correlate with biological age (see FIGS. 3-7). These clocks were generated by selecting the top-n sites based on the absolute value of the coefficients of the full clock (in decreasing order, taking large coefficients first). A linear model explaining chronological age respectively was fitted using the top sites as predictors. Details of the top 3, top 5, top 10, and top 20 clocks are shown in Tables 2-5.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

TABLE 1

| | | Univariant Analysis | | SEQ |
|---|---|---|---|---|
| Site | Co-efficient | (p-value) | Sequence (25 bases upstream and downstream of the 5mC site) | ID NO: |
| Intercept | 11.62197421 | NA | NA | |
| chr1:32205238-32205240 | −1 < x < 0 | 1.47E−36 | GTTTTCACTTCTTGTTCCAGACAGCCGCCTGTTTTTCCGGATCATATTGTTC | 1 |
| chr1:22213697-22213699 | >1 | 1.44E−81 | TGACCCCGAGGGAGCCGCACCCCGGCGCCGCGACGTTCACCTCCGGGGGCGC | 2 |
| chr1:109309639-109309641 | −1 < x < 0 | 3.25E−08 | GCGTCCACCAGGAGCTGGGTGCCTCCGACCTGGCCCAGGACAAGTATGTGGC | 3 |
| chr1:108982935-108982937 | −1 < x < 0 | 2.70E−66 | CTGAAGTCACTCCAGGCTAGAGGGGCGGGGCAATCCCCCTCCCCTGCAATCC | 4 |
| chr1:116329650-116329652 | −1 < x < 0 | 1.39E−16 | GATTGGTCGGGAGGCCCTCGAGAGTCGGCCAGGCTTCGGGGTGCCTGTGGGC | 5 |
| chr1:121182146-121182148 | 0 < x < 1 | 2.96E−18 | CTGGCCCGGAAGGCGCAGGGCATCTCGATGTCGTCTCCCTCCCGGACTGTCA | 6 |
| chr1:118453583-118453585 | 0 < x < 1 | 2.72E−17 | AAGCCACAGGATTGGCACAAGGCTGCGGTGAGGTGGGGGGGGATGCTCAGG | 7 |
| chr1:58324050-58324052 | 0 < x < 1 | 5.00E−49 | CTGCCGAGGGCCGGGGAAGGCCTCCCGGACTCCTCGAAAGCATCTCGCACCG | 8 |
| chr1:65058896-65058898 | −1 < x < 0 | 4.23E−35 | CCCTCCGGCGCGGCACCGTCCCTGGCGGGTCTCGCACAGCTGGACCCTCGGA | 9 |
| chr1:94142224-94142226 | 0 < x < 1 | 0.004658423 | GGCCAAGGACCGCACGTCCCAGGACCGGGTGGAGGTGACGGGGCGCAGCAGG | 10 |
| chr1:98084469-98084471 | −1 < x < 0 | 6.90E−49 | ACTTGTGTTTTTTGTCAACATCCTTCGATGATATGCACTTCCTTAGAATCAC | 11 |
| chr1:100289525-100289527 | 0 < x < 1 | 2.93E−62 | TTCACAGACCGCACACTGAGGCTCGCGGGGCTGCGACCTACTCAGGCCTTCC | 12 |
| chr1:107216628-107216630 | −1 < x < 0 | 4.37E−30 | AACCTGATGAATGAGGCCAGACGGCCGGAAGAACGGCAGAAATAAATGCAGG | 13 |
| chr10:801511-801513 | −1 < x < 0 | 3.10E−56 | GGCCGCGCCAGGACGATGGAGTTGCCGGAGAGAGAGGGGAGCGGCGCCTCTC | 14 |
| chr10:7411293-7411295 | >1 | 1.15E−77 | CAGCACCGGGCATCTGGACAGCAACCGGAGAGCGAGGCGTGTTTCTCCTGGT | 15 |
| chr10:16799192-16799194 | −1 < x < 0 | 3.16E−11 | CTGGACACGGTGGGAGCGTCACAGACGCCACCAGACACTGCGGAGAGGACGG | 16 |
| chr10:18237423-18237425 | 0 < x < 1 | 4.87E−15 | CCGGGGCGCGTGCGGAACCAGCCTCCGCAGAGCCCTCCAGGCCCTACACGCG | 17 |
| chr10:19851621-19851623 | −1 < x < 0 | 2.05E−13 | AGTGGGAGGCCTGGAGACTGCGCAGCGCTTGGTTCTCAGGGGGCCATGCCCT | 18 |
| chr10:20655689-20655691 | 0 < x < 1 | 5.86E−10 | TCCTCTCCCCTCTCCCCAGCCCCCCCGCCGAGGACTAAGGGGCAAGCTAGAA | 19 |
| chr10:44524663-44524665 | −1 < x < 0 | 3.78E−24 | AGCAAAGCAGGCACAATTCCAAGAGCGAGACGATCCCATTCAGCGCTTCTGG | 20 |
| chr10:44524681-44524683 | −1 < x < 0 | 1.33E−19 | CCAAGAGCGAGACGATCCCATTCAGCGCTTCTGGGTACTGTGTCTTGAGGAT | 21 |
| chr10:44846025-44846027 | 0 < x < 1 | 2.79E−19 | CACTCATAATAAAAACATTATACGTCGTGTTTGCCGGAGGATGTCTAAAATA | 22 |
| chr10:40787376-40787378 | −1 < x < 0 | 1.55E−61 | CATCTTGTTGACCAGACTGACCCCTCGGAGCCTAAACCCCCGAGCGGGGCCC | 23 |
| chr10:62719946-62719948 | 0 < x < 1 | 3.34E−78 | CTACGAGGGTCTTTTACCCATTCCACGCCTGAGAAGAGGCCATGACGATCCA | 24 |
| chr10:68649436-68649438 | −1 < x < 0 | 0.156992847 | GCGGCGGGGGTGCTGCGCGGCGTCCCGTGCGGTTGCAGGCTGGGCGGGAGGC | 25 |
| chr11:1992634-1992636 | −1 < x < 0 | 1.20E−14 | ATTCACCACAAAAAGGAATGAAGTACGGACATACACAAGGACGCAGGTAAAC | 26 |
| chr11:33523800-33523802 | 0 < x < 1 | 1.28E−50 | TGTAAGAGGGCTTCGGAGTGGTCCTCGGCCAAGCGCTACCAGCACCGCAAAA | 27 |
| chr11:24476652-24476654 | −1 < x < 0 | 3.10E−09 | ACTGTTCCAGGGTGTACAAATCCCACGCCAACATCCAAAATAATGGCAAATA | 28 |
| chr11:49947821-49947823 | −1 < x < 0 | 7.57E−06 | GTGGATTCAGGATTATACCCCTACCCGACCCCAGACTGTGAGCTGCGGGAGA | 29 |
| chr11:50260436-50260438 | −1 < x < 0 | 2.89E−06 | GCCCCCACCTTCTTGTTTGCCCAAGCGCACCCCCATCCTTGTCCCTCTTCCA | 30 |
| chr11:51974516-51974518 | 0 < x < 1 | 1.68E−19 | CACGTTCATTGACAAACGCGCGGGGCGAAGGCCTAGTGGAGCGCGGGTGGGG | 31 |
| chr11:53576970-53576972 | 0 < x < 1 | 5.55E−76 | GTCCGGCAGCAGCGCGCACCGGGCCCGGCCCCGCGCTCCGGGACGCGAGCGG | 32 |
| chr11:62839836-62839838 | 0 < x < 1 | 3.61E−11 | CACACGGGGCGGGGGGCGCGGTCCCCGGGGCTGCCAGCGCCGCGGTCACTCA | 33 |
| chr12:1207870-1207872 | −1 < x < 0 | 6.31E−45 | CCCCGCCCCGCTCACGCTCACCGCGCGGCGCACCACTCACCGGCTCTCTCCC | 34 |
| chr12:3769194-3769196 | 0 < x < 1 | 1.44E−26 | GTCCCCCCTGCAGGCTCCAGGCTCCCGGACACACTCGGGGTTCAAGTCAAGA | 35 |
| chr12:10418644-10418646 | −1 < x < 0 | 1.59E−42 | CCCCAAAATATCTCCTCTCTGGGTCCGGAAATCCCTCCCAACCCTCTCCGAG | 36 |
| chr12:12684190-12684192 | >1 | 2.80E−21 | CTCAGCCGCTTGGGCGGGTATCCACCGGCCCCGGGGGTAGTCGGCCCCTGCG | 37 |
| chr12:14294419-14294421 | −1 < x < 0 | 1.31E−119 | GTGAGCCAGCCTGGTCAGGGGCGGCCGGAGCCAAGGACCCGTGGCTCCCTCA | 38 |
| chr12:46491407-46491409 | −1 < x < 0 | 1.86E−60 | CCTCCTAAACGTGGGCCCTGTGTTCCGGAGCCCCTGGAATCGTTTCAGCATG | 39 |
| chr13:3737760-3737762 | −1 < x < 0 | 5.17E−41 | CGCACCTGACCCCCGTTGCCCACTGCGAAATTTCTCCAAAGCTTCCATCCCA | 40 |
| chr13:10113845-10113847 | −1 < x < 0 | 2.88E−55 | CGGGGCACTCCCTGGACAACAGGGACGCGCTGGCCCCAACGTTAGGCGCAAT | 41 |
| chr13:37195182-37195184 | 0 < x < 1 | 2.76E−05 | CCGACACCTGCAACAACGGCACCTGCGGGGGGGGAGGGGTGTCGGCTGGGCG | 42 |
| chr13:37332032-37332034 | −1 < x < 0 | 3.16E−13 | AGTCCAGGGAACAGGGCCAAGGTCACGCAGAAAGGTCACTGTTATCAAAACG | 43 |
| chr13:37474592-37474594 | −1 < x < 0 | 2.27E−44 | TTCCCGGCTCCCGAACCAGGGTGGCCGAGCCAGGCCTCTGCCTCCGGGGGTC | 44 |
| chr13:37665667-37665669 | −1 < x < 0 | 1.49E−32 | CCCTGGTGGGGGCCTTAGGCAAAAACGGGTAGCCGCAAGCAGGGACGCAGCG | 45 |
| chr13:37877759-37877761 | 0 < x < 1 | 6.31E−36 | TGCAGCCGGGCCTCCTGGGGCAGGGCGAGCTCGCACCTGCTGGCCTCCCCGC | 46 |
| chr13:37906098-37906100 | 0 < x < 1 | 3.77E−59 | TGACGCTCCCGCGGCGCGGCTCCCCCGCCGAGCCCACGGCGCTCCGGCCCCA | 47 |
| chr13:38339807-38339809 | 0 < x < 1 | 7.26E−80 | CTCGCCTGCGCTCCTCACCTGGGCGCGCTGCGCGGTCACGGCAGATGCTCGG | 48 |
| chr14:345761-345763 | −1 < x < 0 | 1.22E−06 | GGGGCGACCCCTGGACTGTGGCTGGCGTCTGCTGTGAGGTGGCCTGTGGGGT | 49 |
| chr14:6144664-6144666 | 0 < x < 1 | 6.62E−07 | TATCTCAGCCATTCATAGCGCCTAACGGCTCACAATACCCTAAAGGCTCCCT | 50 |
| chr14:5949450-5949452 | 0 < x < 1 | 9.32E−81 | CCGGCGGTGCCAGCATGTCCGCCGCCGTGGCGTGCCTGGACTACTTCGCCGC | 51 |
| chr14:7586529-7586531 | −1 < x < 0 | 6.72E−09 | GAGCTTTACCTCCCACCACTCCCTCCGCATGTCCTGGGCTCTGGCCATTACC | 52 |
| chr14:39735273-39735275 | −1 < x < 0 | 3.33E−07 | TGGTAGCTGGGGCCCCCGCCTGGGCCGCCGTCTCCGCCGCCCGGGCCGCTGT | 53 |
| chr14:58424689-58424691 | −1 < x < 0 | 1.56E−75 | TTTCTAGGACTACGTCTGTGCACCTCGTGTGGCAAATAAAACTATGCTAACA | 54 |
| chr15:2611795-2611797 | 0 < x < 1 | 6.88E−35 | CCTCTAGACCCCAACCCTTCGAGGTCGGTCTCAGGTCCGGTCTCAGGTCTCG | 55 |
| chr15:7297280-7297282 | 0 < x < 1 | 6.12E−77 | AATAACTCGTCAAGTCCCTGGGCTCCGAGGCAGCCTGCCTCCCCACCAGCCC | 56 |
| chr15:10856498-10856500 | | 7.42E−18 | CCCCAGCCGGGCCGGCTCGGGTTTCCGACGGAGGGCCGGGCTGCGCGGCGTC | 57 |
| chr15:10856612-10856614 | 0 < x < 1 | 1.44E−59 | GGCAGCCGGGGCAGCGGGAGGCGTGCGCAGCGCCGGCGAGGAGGACCCGCG | 58 |
| | | | C | |
| chr15:18190829-18190831 | 0 < x < 1 | 0.032879523 | CACCGCTGAGCTTCCCGCCGCCCTGCGCAGCGCAGCCCTCTCGCCGCTTATA | 59 |
| chr15:20380665-20380667 | −1 < x < 0 | 1.86E−07 | AGTTTCTTTTTGGGAAGATGAACCCCGACGGAGGCAGCTGCGCGGGGTGTGT | 60 |
| chr15:51319029-51319031 | −1 < x < 0 | 1.03E−36 | GAGTTTCTGTGCACGAGGTCATCCTCGGTCAAGTATTATTCCCACCCGTCTC | 61 |
| chr16:8886356-8886358 | >1 | 2.94E−15 | CCGGCCGGCCGGCCACGGAAACCTCCGGAGGCCCGGGCCCCGCCGGGGTGTC | 62 |
| chr16:8886545-8886547 | >1 | 2.03E−101 | GGCGGGCTCCCCGTGCGCGCACGGCCGCGGCCGACCTCTCCGCGCGGGCTCC | 63 |
| chr16:15250294-15250296 | −1 < x < 0 | 5.04E−14 | CATAACTGATGCCCACAGGGTCTGACGTTCTGAGTTAACCTGCATTATAACT | 64 |
| chr17:15014527-15014529 | 0 < x < 1 | 2.86E−29 | ACGCGGCATAGCGGGCTCCGCGCTCCGGCAGGGGTCGGTCGTCGAAGGTCGG | 65 |
| chr17:19405682-19405684 | 0 < x < 1 | 1.09E−17 | TGGATAGAGAGGGGTCCCTTAAAAGCGTGCCTTGAGTTCAGGCTTTGAAAAA | 66 |
| chr17:33544113-33544115 | 0 < x < 1 | 3.12E−75 | CTGGGGGCTACGAGCGAGAATCCCTCGACGCTCCTGAGAAACTCAGACCAGA | 67 |
| chr17:33851462-33851464 | >1 | 2.82E−131 | ACAGCAGGATGAAGCCGAAAGCCTCCGAGTGCCGGTCGAAGAAGTACTCGTT | 68 |
| chr17:34115428-34115430 | −1 < x < 0 | 3.35E−49 | TCCTGGGCCCGCGGCGCTGGCCCCTCGGGCCTCGCTGAAAGCCCCTTGTTCC | 69 |
| chr17:48274513-48274515 | 0 < x < 1 | 1.03E−43 | ATGCTACTGCAGGCACTGGCTGCTTCGGCGTGGCTCTGTGCGGTAGTGTCTC | 70 |
| chr17:49222883-49222885 | 0 < x < 1 | 7.66E−32 | GAAGCGAGGAGCCGCCTCCCGAGGCCGGTCCCGGCGAGCAAGGGCGCCTCGG | 71 |
| chr17:55836014-55836016 | 0 < x < 1 | 1.59E−15 | CGGGTGTCCTGTGTGTATCTGTGTGCGTGTTTGTTTAATCCTCCCCCAAGCT | 72 |
| chr17:60022289-60022291 | −1 < x < 0 | 5.73E−66 | ACAATCCTTTTAAGGCAACGTGTCCCGCCTCTCCTAACCATATAAGTGCAAC | 73 |
| chr18:5425112-5425114 | 0 < x < 1 | 7.01E−67 | CAGACGCGAGCGAGGCCGGTGGAGGCGGGGTGGCTGCGTTGGACGGGACGG | 74 |
| | | | G | |
| chr18:31999739-31999741 | 0 < x < 1 | 1.47E−20 | GGTGCCGTCGGCGAAGCGGGCCAGCCGGTTGGAGCTGCGCCCGCAGCCGCCG | 75 |
| chr18:46670535-46670537 | 0 < x < 1 | 3.67E−30 | GTGGCCTGGGCAGGTCAGCGGAGCCCGCGGCGCTCAGCGCCCCACCTGTATG | 76 |
| chr18:46990963-46990965 | 0 < x < 1 | 7.61E−44 | GGCGCGATGCGGGCGGCCGCGGCGGCGTCTACGAGCACCTCGGCGGGGCGCC | 77 |
| chr18:44679236-44679238 | −1 < x < 0 | 1.20E−31 | TCCCGGAAGTCCTGCTCAACTAGCCCGGCTGGCGGCCCGGTGTCACCCGCTG | 78 |
| chr18:44686394-44686396 | −1 < x < 0 | 2.56E−78 | GTGCCACCATCCCCGGGTTGAAGGACGGGTTCCGAGTGAGCCAGGAACCACC | 79 |
| chr18:48681702-48681704 | −1 < x < 0 | 1.01E−08 | GGATCCGTCATTGACCCTTGGAGAACGGAAGATTCTGGGGGCTCAAAGGAGT | 80 |
| chr18:50259206-50259208 | −1 < x < 0 | 5.46E−54 | CCAACATGACGCTCATCGGAGAGAACGGCCACAGCACCGACACGCTCACGGG | 81 |
| chr18:51915009-51915011 | −1 < x < 0 | 0.06485116 | TTAAGCCCACGCTAATTTCCATAGGCGAGTTAGAATGTGGCCTAAATTGCTT | 82 |
| chr18:51374197-51374199 | 0 < x < 1 | 7.00E−102 | CCCACAGCCCCTCGCGTCCCCGGAGCGCGCACGCCCGCAGCGGTGCAAACTG | 83 |
| chr18:53063592-53063594 | −1 < x < 0 | 6.71E−33 | AGAAGGATCTGGAACGTGTAGGAGACGTGCAGCTTCTCAGGGTCCGTCTCCA | 84 |
| chr19:44679975-44679977 | 0 < x < 1 | 1.44E−18 | GCCCATCGCCTGCGGCCTCGGTCTCCGGGGAGAGGGAAAGTAACTCCGGAGC | 85 |
| chr2:32494387-32494389 | >1 | 0.178817525 | TTGGAAGCTCGGGGGGCGGAGATCCCGCGCCGCCCGGCCGGCCTCGGGGCGG | 86 |
| chr2:31655107-31655109 | −1 < x < 0 | 1.12E−44 | CTGCACAGCCCTGCTCCGCCCTGCGCGGCCCTGCACGGTGCTGCTCGGTGCT | 87 |
| chr2:32780982-32780984 | 0 < x < 1 | 2.44E−08 | TTGAGTGACGGCTAGCGGCCCGCCCCGGGCGTGACGTCATCCCGGTGTTGCT | 88 |
| chr2:64665545-64665547 | −1 < x < 0 | 2.99E−09 | CTTCACAGTAAGCCTAAAGAGTAGGCGTTATCTCCGTTTATGGTGTAACTGG | 89 |
| chr2:65566948-65566950 | −1 < x < 0 | 3.68E−14 | CGCCAGCCTGCAGCAGGTCCATGAGCGGACGTGGGCCCGGCAGTTGCTGCAG | 90 |
| chr20:3395529-3395531 | −1 < x < 0 | 1.27E−18 | AAAAGCCAGGGGAGAGGGGAGGCGCCGGCTCCCACAGGCCACTGTAAGTCCC | 91 |
| chr20:3833805-3833807 | −1 < x < 0 | 4.84E−49 | AGTAAGGAGCTGCTCTCATGACTGTCGTTGCTGTTGCTAAGTGGCCCTTTTC | 92 |
| chr20:3832009-3832011 | −1 < x < 0 | 8.18E−17 | CCGCGGCCCCACTCGGTCACGCCCACGGGCACCCTCACGGAGCCTCCAGGGA | 93 |
| chr20:18594283-18594285 | 0 < x < 1 | 2.07E−50 | CCCGCGCGCTGCCGCGGCCGCCTGTCGGCCAAGGAGCTCAACCACGTCCTGC | 94 |
| chr20:36046282-36046284 | 0 < x < 1 | 8.06E−63 | CTTCATTGCATCGCCTTCCCGACGCCGGGTGCCAACAAGCAACCAAAAAATT | 95 |
| chr20:40374930-40374932 | 0 < x < 1 | 4.41E−91 | CCAGGATTCCACATGGTCAGGAGAACGCTCTCTGCACATCATGGACACCAAA | 96 |
| chr20:44689213-44689215 | 0 < x < 1 | 3.92E−125 | TTCTCCCGCCTCAGCACCTCCAGCGCGGACAGCTCCTCGCTGTCGCCTCCAC | 97 |
| chr20:44942213-44942215 | >1 | 1.98E−31 | GGCAGGGCCGCCAGGCGCCGGCCCCCGGTGAAGAAGTCCTCGGCGCTGCGCT | 98 |
| chr20:45091614-45091616 | 0 < x < 1 | 5.38E−50 | TTGCGTAGGAGGTAGGAGGCGAGGCCGTGGGGCTGGGAACCCGAACTGGGCT | 99 |
| chr20:45360920-45360922 | −1 < x < 0 | 1.11E−19 | CAAGTCACCCAGCTCATCTGTTGAGCGTTCACTGTCCTAAGCCCTTTATGAC | 100 |
| chr20:46098568-46098570 | 0 < x < 1 | 4.57E−68 | TCGGTGCCGCCCGCCTCGGGCTCTGCGCGCGGCCAGCGCTGCGGGTGACAGA | 101 |
| chr20:49844561-49844563 | −1 < x < 0 | 2.37E−33 | TGAAATTTTCGAGTCTCGGAGACGGCGTGGATGCTGGGGGTCCTGAAAGCCT | 102 |
| chr20:49940551-49940553 | −1 < x < 0 | 4.34E−59 | ACCTGCCCATGGGGGATAATAATAACGTTAATGAAACCCTTGCAGAGTACTC | 103 |
| chr20:54301453-54301455 | >1 | 3.30E−119 | ACCACGCCCCGGGGGCCGAGCGTCGCGGGGGTCGGACGCCGAGCAGCTGCCG | 104 |
| chr20:53921003-53921005 | −1 < x < 0 | 9.56E−10 | CCCTCAGGAACTCCTCCAGGTCCAGCGTCCCGCTGCCATCACGGTCCCAGCG | 105 |
| chr20:57347921-57347923 | −1 < x < 0 | 1.16E−62 | ACCAGCCGCCCAGCCGTGACCTTTCCGGGCCTGTTCCTGCGGCTCTGGGCGC | 106 |
| chr20:57318012-57318014 | 0 < x < 1 | 1.45E−14 | GCGGCAGCTCGGGAAGTCAACCCCCCGGCGACATCTGACAGGGCAGGCCGGC | 107 |
| chr20:57621119-57621121 | −1 < x < 0 | 2.51E−16 | AGCGCAGGACACCCCTGCACGTCGGCGCCGCGGGCGGTGCCAAGGTCCTGGG | 108 |
| chr21:16233628-16233630 | 0 < x < 1 | 1.28E−11 | CGGCGGGCGCGCTGCTGGCGGGCGCCGAGGGCGGGGACGTGCGCGAGGCCAC | 109 |
| chr21:41864188-41864190 | −1 < x < 0 | 1.42E−10 | AGAATAAGTCCGTCTTCATCCTGGGCGCCAGCGGGGAAACCGGCAGAGTGCT | 110 |
| chr21:43490796-43490798 | >1 | 3.27E−88 | ACGCGCTGGCCCGCGCTGCCCGCCGCGCTCGGGAGAGCATCCCTGCCGCTGC | 111 |
| chr22:46374563-46374565 | >1 | 1.65E−07 | TTGGACGCGGAGGCCGTCCGGGCGCCGAGCAACGAGCAGCACAGCACGGTGG | 112 |
| chr23:11942623-11942625 | −1 < x < 0 | 1.81E−08 | ATCCAGAGGGGGCTCCTGGGGCCTGCGTTGCCGGAGCACCCCAAGCAAATAC | 113 |
| chr23:29966538-29966540 | −1 < x < 0 | 2.56E−38 | AGGGGGAGGTGACATGGGAATTAAACGCCCTTTCCCTCAGCCAGTGCCAAAT | 114 |
| chr23:37987526-37987528 | −1 < x < 0 | 0.15833084 | CGGGGCGGGAATCCGCCCGCCCGCTCGCCCCCGGCGCCTAGACCCGGGGCGC | 115 |
| chr23:50123623-50123625 | 0 < x < 1 | 8.61E−122 | CCCAGAAACTACGGCAAACTCGCGCCGGGTGCGCGCTGGAAGGGGGAGTCCG | 116 |
| chr24:12518910-12518912 | >1 | 3.37E−125 | CTCTTCCTCGGAGATCCATGCTCGCCGTGCAGCGAAGGAGGCCAGATGTGCT | 117 |
| chr24:17354043-17354045 | −1 < x < 0 | 9.00E−50 | TGGGTAGGTGAGAGCAGGGCAGGGGCGCCAAGTTCGGGCTGCCCAGACTGCT | 118 |
| chr24:21051024-21051026 | >1 | 1.02E−124 | ACGTTGCTCTTCGTGCTCCTGCCCTCGCTGGTCGTGCAGCTGCTCAGCTTCC | 119 |
| chr24:30860619-30860621 | >1 | 0.176671196 | CAGAACCGCGCTTCCCCGAAACTCGCGGGCGGTGGGATCGCAAGTCACCCCC | 120 |
| chr24:24968751-24968753 | −1 < x < 0 | 3.88E−104 | GTCCTCTGGCGAGGGAAGTTACAGACGCCAAGCCCAGAGTCTGGGGTGCCCA | 121 |
| chr24:43790837-43790839 | 0 < x < 1 | 9.16E−44 | CGCCGGGCCCCAGGAGCCGCCGTCACGACCGCCGCCTCGCGTAGCGCCTCCG | 122 |
| chr25:15332071-15332073 | −1 < x < 0 | 2.20E−31 | ACCACCGACAAAAGATAATGTTGCCCGAATAAGTTACGTGCATCTTAACATG | 123 |
| chr25:15332101-15332103 | −1 < x < 0 | 1.11E−28 | AAGTTACGTGCATCTTAACATGTCACGGGATCTGTTCCTGTGTGTTCTTGTG | 124 |
| chr25:16764068-16764070 | −1 < x < 0 | 1.40E−42 | CGCTCGAGCATCTACTCTACTGCGGCGAATTTAATCCACATTTCCTCTGCAT | 125 |
| chr25:18016409-18016411 | 0 < x < 1 | 3.89E−62 | AGAGCCAGGCGCGGCCCCGCGAGGCCGGGTGCACGTGCACACGAGGGGCGCC | 126 |
| chr25:35778245-35778247 | −1 < x < 0 | 1.05E−28 | CTTGAGGCCTTGAGGATGGCCCCTGCGTCCACGACCAGGGCTGGGGCTCCTG | 127 |
| chr25:47511184-47511186 | 0 < x < 1 | 1.37E−79 | CCGGGACCCGGAACGCAGGCGCCGCCGACCCTCAGTCCCGGGCCGCGTGCAT | 128 |
| chr26:5490242-5490244 | −1 < x < 0 | 3.09E−113 | TCCCAGCGTTTCAGCTCCTGAGTGTCGGCTGGAAGCCCGCCAGGGTTACCAT | 129 |
| chr26:9207175-9207177 | −1 < x < 0 | 1.73E−06 | CCCGGCAGTACTGGCTCTTCGCCCGCGAGCACCCGCACCACGCGCCGCTGCG | 130 |
| chr26:10691576-10691578 | −1 < x < 0 | 5.16E−17 | TGTATTAACTCCATAAATGTATATACGGGATAGAAGGACCTGCCCTGTCTCC | 131 |
| chr26:22918623-22918625 | −1 < x < 0 | 3.10E−26 | TAATTCCATTCTGGTTTGTAGAATTCGTCTGCAGGCTATTGTGGATAGGATA | 132 |
| chr27:9490065-9490067 | −1 < x < 0 | 7.50E−09 | CCCTGAGCAGTGCCACCCTTCTTCCCGGAAAAGGAGGGAAGCCATCCTTTTC | 133 |
| chr27:29307097-29307099 | −1 < x < 0 | 2.59E−25 | CAACATGCAACTCGGGGTTGTTCTGCGGATGTGTTTAAGGCCAATGTAATTC | 134 |
| chr27:45037864-45037866 | 0 < x < 1 | 4.82E−106 | CCAGCGGCCGCCAGCCCTGCGCCGCCGCGACACAAAGAGTGCGGGCGATTCC | 135 |
| chr28:8994532-8994534 | 0 < x < 1 | 4.42E−57 | GAGCCCGCCAAGTTAGAAGACTGGTCGGTCGGGGCCACACGGCTGGTCCGGG | 136 |
| chr28:21744238-21744240 | −1 < x < 0 | 1.81E−57 | TAGTAGTCGATAGAGGACTGGAACACGGCACTTCAGATAAAGAATGCTCCAG | 137 |
| chr29:9541477-9541479 | −1 < x < 0 | 4.37E−13 | ACTAGATTTTGAATTCTATGATGACCGGGACTACTACTACTTCTACCTTTGC | 138 |
| chr29:19479611-19479613 | 0 < x < 1 | 6.53E−89 | TGGGGAGGTGGGGAATCGCGTACCACGGTGGCAGCTTGCTGTACCAGGGCAG | 139 |
| chr29:32189910-32189912 | −1 < x < 0 | 2.95E−66 | GCAGGTGGTGGGACAGCCATTGGGCCGTGTAGAACCGAGGTTCCCACGTTTG | 140 |
| chr29:35629292-35629294 | −1 < x < 0 | 3.46E−55 | TCCCGATGATGGTGATTGATCATTACGAGGACGGTAATAAGATTGTTACCCG | 141 |
| chr3:17063027-17063029 | 0 < x < 1 | 7.60E−31 | CCCGCCCGCACTCCCGGCCCGCGCCCGGAGGCAGTGTGGGGTGCGGGCCTCC | 142 |
| chr3:30303462-30303464 | 0 < x < 1 | 1.61E−47 | TGGAGCGGTGCGCGATGCTGTCTCACGCCGACCTCCTGGACGCCAGGCTAGG | 143 |
| chr3:30303526-30303528 | 0 < x < 1 | 2.78E−23 | TCTCGCCCGGGCAACCAGGAGAAGCCGGGGCAGGAAATTGTCAATTTGTTCC | 144 |
| chr3:31677748-31677750 | 0 < x < 1 | 2.45E−111 | GCTGAGCCCGCAGCTGCGGCGCGGACGCCGGTCGCACGCCCGGCGGGGCTGG | 145 |
| chr3:46852141-46852143 | 0 < x < 1 | 3.72E−16 | CTATGGGCGATATTGCAAAGCAAGACGACCCGAGGCACAGCCAAATTCCACT | 146 |
| chr3:46843750-46843752 | >1 | 1.98E−135 | CCAGGGCCACAGACCTGGAAGTCGCCGGGAGCCCGGCAGCACTCCTCTCAAG | 147 |
| chr3:70831746-70831748 | 0 < x < 1 | 3.57E−19 | CTACCTGTCGGCGGGAGGCGAGCACCGGGCGCCCGCACGTCGCGCCGGCGGG | 148 |
| chr3:72353348-72353350 | −1 < x < 0 | 9.65E−58 | GGGACACCTACTCTGGGCCCCGGCACGGCCGAGACAGACAGGTGCCTGCAAA | 149 |
| chr30:8031058-8031060 | 0 < x < 1 | 1.27E−55 | GGCCGTCGGGCGGGGGCCGGCTCACCGCGGCGCGCGTGGGAGGGCCGGGCCG | 150 |
| chr30:8031060-8031062 | 0 < x < 1 | 1.06E−40 | CCGTCGGGCGGGGGCCGGCTCACCGCGGCGCGCGTGGGAGGGCCGGGCCGGC | 151 |
| chr30:37920883-37920885 | 0 < x < 1 | 3.52E−27 | TTCCAAGGGCGCCGGGCCGCCTCCGCGCGTGGAGCGTCCACTGTTGGGGTAA | 152 |
| chr31:35123688-35123690 | 0 < x < 1 | 2.86E−15 | CACCCTCCGGACACCCCAGGGAGCACGGACAGGTCTTCCCAGCACGGCAGGC | 153 |
| chr32:10044870-10044872 | −1 < x < 0 | 1.29E−11 | GTGAGACGGAATGTGTGAGTATGTACGTAACAGGTTTTAAGAAATTGTGTAC | 154 |
| chr32:40409729-40409731 | 0 < x < 1 | 2.89E−62 | GAGCTCTTCAAGGGCTTCGTCAACGCGGTCGCCGCGGTGCGCCAGGACCCCG | 155 |
| chr32:40409736-40409738 | 0 < x < 1 | 1.49E−50 | TCAAGGGCTTCGTCAACGCGGTCGCCGCGGTGCGCCAGGACCCCGACGGCAC | 156 |
| chr32:40571018-40571020 | −1 < x < 0 | 3.33E−83 | TTTGGGTCTTAGCCAAGGTACACATCGGCGTTTGTTTTGCATTTGTGATCTA | 157 |
| chr33:23247947-23247949 | −1 < x < 0 | 1.70E−32 | GACAAGGGTATGGAATACCTACAAACGGTGATTCTGATTCTTTTCAAGGGTG | 158 |
| chr33:23473772-23473774 | 0 < x < 1 | 1.07E−05 | ACAGGCAACATCGTGGGCCTTCTACCGGGCACTTCCTTCTTCTCTGCCCGCA | 159 |
| chr33:23473856-23473858 | 0 < x < 1 | 7.19E−11 | GCCCAGAGGTATCCACAGCCCAGCTCGCTGTGGTCTCACACACAGCGTCTCA | 160 |
| chr33:26512692-26512694 | >1 | 3.39E−65 | CGGCTGCCGCGCCCGCAGTCGTCGCCGCCGCGCTCCTGCCAGGCCGACCTGG | 161 |
| chr33:26512711-26512713 | >1 | 3.97E−30 | CGTCGCCGCCGCGCTCCTGCCAGGCCGACCTGGAGCGGAAGCTGAAGCCGAA | 162 |
| chr33:19810904-19810906 | >1 | 1.38E−70 | GCGCTCCTCGCCGCTCTCGGTGGCCCGGAGAACAAAAGCCCCGGGCCTGGCC | 163 |
| chr33:28241609-28241611 | −1 < x < 0 | 4.28E−75 | GGAGGGCAGCTGGGAGTACTTCCCACGCCCACATGAGCCTTTGTGTGAAAAT | 164 |
| chr33:28241833-28241835 | −1 < x < 0 | 1.22E−83 | AATTACTTTCACTTTGTTGTGAGGTCGCTGCTGTCTTCTGTGTCACCCCAGG | 165 |
| chr33:29201592-29201594 | −1 < x < 0 | 2.23E−80 | CTGGTGCCCGACGGAAATAGGAGGCCGGCAGCCTGCCAGAGTGGGACAGTCC | 166 |
| chr33:29201578-29201580 | −1 < x < 0 | 4.79E−64 | GTGGCCCTGTTACCCTGGTGCCCGACGGAAATAGGAGGCCGGCAGCCTGCCA | 167 |
| chr34:21237427-21237429 | 0 < x < 1 | 1.02E−35 | GGGGGTCGCTTGGCTTGGTTGCCCGCGAAGAAGGAGTTGGTGTCCTGCAGGC | 168 |
| chr35:7689584-7689586 | 0 < x < 1 | 5.64E−14 | GGGCGGGGCCTCCCGGGCTGGAGTGCGGTACCTGAGCGAGACGCGCACCAGA | 169 |
| chr35:23852932-23852934 | >1 | 1.76E−44 | ACACGCCGCTGCGCTCCTTGGACGACGACACGGCCTGGACGATCAGCTCCGA | 170 |
| chr35:23853763-23853765 | 0 < x < 1 | 2.09E−70 | GCTGACGCCGTCCTTCACGCGCGGGCGGCACGTGTGGAAAACGCAGCGCGCA | 171 |
| chr36:18662589-18662591 | −1 < x < 0 | 2.78E−36 | CCCGGTGGTCTCCAACACAGCCAGGCGTCGGGTTTTGTGCGGGATGGTGCAG | 172 |
| chr37:2007181-2007183 | −1 < x < 0 | 7.34E−48 | TTCATCATTGGATGATAGTTTTTCACGACCTTGAGAGTCTCCTTGTAAAACT | 173 |
| chr37:26735403-26735405 | −1 < x < 0 | 3.70E−30 | CCCTTTTTTACACATGAACAAAACGCGCTACAGAGAGGAATAACCTCCCCAG | 174 |
| chr38:14762928-14762930 | 0 < x < 1 | 1.11E−57 | GCCACGCGGCCCGCGCCCAGCCCCACGCACAGCGACAGCAGGTCCGACAGCA | 175 |
| chr38:21317970-21317972 | −1 < x < 0 | 1.16E−21 | GGAGTCTAGGATGGCAGTGTGACTCCGAGAAGGTAGGACAGCGCGGGAGAGG | 176 |
| chr4:19870655-19870657 | −1 < x < 0 | 2.31E−06 | CCTAAATAAACTCTTAGGAATTCCCCGGTTCCTTCTCCCTGTCCCCACTGCA | 177 |
| chr4:23880514-23880516 | −1 < x < 0 | 0.018537335 | CGCCCGACCCCGGAACCCCCGGTGGCGTCACTGCTGCGCGTCCGCCGGAGCA | 178 |
| chr4:25509815-25509817 | −1 < x < 0 | 1.33E−39 | ATGGCCTCCTGTCTATCCTGGGACCCGTCTCCACCCCCATCAGAGCCAATGA | 179 |
| chr4:34981731-34981733 | 0 < x < 1 | 4.21E−25 | TGCGCCCCGGGGTCTGGCTCGTCCGCGCGGGGCAGCGGCCCATGGTCGGCGG | 180 |
| chr4:36661557-36661559 | −1 < x < 0 | 5.86E−25 | TATTACAGTGTCGTCGAGTCAAACGCGCTTTTCACTCAATGGAAATTCCTCT | 181 |
| chr4:36180013-36180015 | 0 < x < 1 | 1.08E−119 | GCGGGCTGGGGGCCCGGAGGGGCCGCGCCTCGCACCGCGGACGCTGCAGTTC | 182 |
| chr4:40697330-40697332 | 0 < x < 1 | 3.06E−42 | GGGAGGGCGGCTGCTCGGGTCACTCCGGGGGCAGCATGCGCGGCGGCGCTGG | 183 |
| chr4:43217432-43217434 | 0 < x < 1 | 3.97E−81 | GTGTCGTGCACGTTAGCGTGGCCGACGGCGTGGTGGGCCCTGGGCCGCAAGC | 184 |
| chr5:19192793-19192795 | 0 < x < 1 | 6.11E−87 | TAGCAGGGCTCTGGGAGTCCCTAGCCGGTGCAAAGTGTTAGAAAGACATCAC | 185 |
| chr5:21480151-21480153 | >1 | 4.09E−32 | TTGAGCTGCCTGGAGCAGCAGCCGTCGGCCACCTAGCAGGAGTGTCCTCGCA | 186 |
| chr5:32347978-32347980 | −1 < x < 0 | 0.890183588 | ACACGATAGCCCCTGGCCCGGTCCCCGGCCACCCCCGCCAGCAGCCCGATGC | 187 |
| chr5:32612576-32612578 | −1 < x < 0 | 1.82E−15 | ACTTCCTTGGTCAGAACCACGGCTTCGCCTGAGAACTAATTGTTACCCTCGA | 188 |
| chr5:32711703-32711705 | >1 | 0.068664987 | GGTATCCCGTCCCCTCGGCTCGGCTCGGCTCGGCTCGGCTCGGCGGGCCCCG | 189 |
| chr5:32884319-32884321 | −1 < x < 0 | 2.18E−34 | CCAGCTTTGTTTGGGTCTCCCACACCGCCTGTGTATGAATGGGACTAAATTT | 190 |
| chr5:40054086-40054088 | 0 < x < 1 | 7.25E−31 | CGTACAGCGCGTCCTGCGCCTCCAGCGGCATGGCCGGGCCGACCGGCCCCGG | 191 |
| chr5:33220460-33220462 | 0 < x < 1 | 2.71E−47 | ACGTAAGGTGAGAAGGCGCGAGCGCCGCGGACCCGTCCGGTGGGATGAGGGG | 192 |
| chr5:46881938-46881940 | 0 < x < 1 | 6.21E−74 | TCCTCGGCCGTCAGCACCGTCTGGCCGGACATGTCGCTGGCGCTGCCGCCGC | 193 |
| chr5:55848493-55848495 | −1 < x < 0 | 1.84E−13 | GACCCTGTGCCCTCAGCTGGCCTCACGTGCATGGAATGGTGCTGTGCCCTGT | 194 |
| chr5:56981423-56981425 | −1 < x < 0 | 9.11E−71 | CTGCCGGGCCTGGCCGGGGCCGCTTCGTGGTCTGCACTTACCCCACGTGCCA | 195 |
| chr5:61645225-61645227 | >1 | 1.61E−64 | TAAAGGGGCGATGCCGCGACCCAGGCGCTGGCGTGGGGTCCACCGGCGTCGC | 196 |
| chr5:64909470-64909472 | −1 < x < 0 | 2.94E−20 | AGAGTGTAGAGCTGCTGGGGGCTGCCGTCTGCCCTGCTGGCCTGCTGTGGTA | 197 |
| chr5:76278186-76278188 | −1 < x < 0 | 3.25E−40 | TTCATCTCTGGTTCTTGTCAATCCCCGCAGGAACTGTGTCCTTCAGACACAG | 198 |
| chr5:76278307-76278309 | −1 < x < 0 | 2.57E−05 | GTGAAATTCCTGCAAGTCGTGTTGCCGACATAGTCTGCAGGACAAACTTTTA | 199 |
| chr6:8231142-8231144 | −1 < x < 0 | 6.01E−28 | GAGAGGCGGGCCCCTGGCTTTTGGGCGCAGACCTCCAGGGCCCGCATCAAGT | 200 |
| chr6:8915114-8915116 | −1 < x < 0 | 3.25E−61 | TCCAGACCCTAGCAGTTGGAGGATCCGAGTTCGAATTCTGTCTTTGCCATTC | 201 |
| chr6:29756505-29756507 | 0 < x < 1 | 1.70E−24 | TGCACGACCACCGCAGCGTCCCAACCGTGGTCCTGGCCCCTGGCTCCCTCCT | 202 |
| chr6:31453418-31453420 | 0 < x < 1 | 4.35E−06 | GCGGCAGAAGTCCGGCGTGGGCGCCCGGGTCGGGGGCGCGTCCGAGACCCCC | 203 |
| chr6:40418271-40418273 | 0 < x < 1 | 3.39E−53 | GGCCCGATTGTGCCGCCTGTTGTGTCGGTTGACTCTTGCCCGATTGCCGCGC | 204 |
| chr6:40624669-40624671 | −1 < x < 0 | 7.26E−43 | CCTGGTGCCGGAGAGCGGGATGTCACGGCTTGTATCGATCACGCGTTGAACC | 205 |
| chr6:41485415-41485417 | −1 < x < 0 | 3.69E−05 | GGCCGTCCATGGCAGTGCCGGCCCACGAGGCAGGGGGCAGATAAGGGGGCCG | 206 |
| chr6:41661247-41661249 | −1 < x < 0 | 3.49E−53 | TCTCTGGCGCAATAAAAGAGACCCCCGTGAGGAAGCCCCCCCAGCACTCATA | 207 |
| chr6:45773846-45773848 | >1 | 5.94E−243 | GAGAAGGACGTCCCTGTCGCGAAGCCGTTCGCAGCGCGCGGCCCTGGGGGCG | 208 |
| chr6:60675998-60676000 | >1 | 1.02E−37 | GATAGCTTCCTCGGCTCGGTCGGGGCGGCTGCTGAGCGGCGGAGCGGCGCGG | 209 |
| chr7:388496-388498 | 0 < x < 1 | 3.63E−26 | GAGGGGACCTGCCCCGTGGGGTGGCCGTGCAGGTTCGGGGGCGCATCCTGCG | 210 |
| chr7:38420583-38420585 | 0 < x < 1 | 6.52E−09 | CAGGGACCTCGGCCCTCCCTCCCAGCGGCCGGGACCAGACTCCGGGGTGCCT | 211 |
| chr7:38136719-38136721 | −1 < x < 0 | 1.74E−12 | GCGTCCCCGGAGGTGTTTACCGACCCGCGCACACACACCCCGCTACGTGCCA | 212 |
| chr7:41785316-41785318 | −1 < x < 0 | 3.46E−24 | AGGGCTGGGAAGATTGGCAGCCCTTCGGAGTTTATGCGTGAAGGGAGTCCAT | 213 |
| chr7:41785327-41785329 | −1 < x < 0 | 1.25E−17 | GATTGGCAGCCCTTCGGAGTTTATGCGTGAAGGGAGTCCATCTTACCCTCCC | 214 |
| chr7:41980137-41980139 | −1 < x < 0 | 1.31E−49 | CCACAGCATCAGCCCAGGAGAGGCACGGTTTATTGACAGGTTACACCCCAGG | 215 |
| chr7:42504060-42504062 | 0 < x < 1 | 2.99E−18 | CCACAGCGGAAACCATCACTGGCACCGGCTACTCCTTACGGACAATGCTGGG | 216 |
| chr7:78016807-78016809 | 0 < x < 1 | 3.99E−20 | AAATGTACAGCCAGCGATTTGGCATCGTACAGCGGGAGGTTAAGGGCCCCAC | 217 |
| chr8:14013084-14013086 | 0 < x < 1 | 1.77E−07 | ACGGAGAAACCACTTTATCTGAGGACGGTCGAAAATTAGATCCTACTTTCCC | 218 |
| chr8:14259939-14259941 | −1 < x < 0 | 1.79E−36 | ATTTTAGATGCCAATTCTTTGAAGGCGTTGGATGTAACACCCCTTCATTTCC | 219 |
| chr8:4291048-4291050 | −1 < x < 0 | 2.83E−89 | GGCGGGCAGGTAGAGGGCCGATCCCCGGCCTTTTCACATCCCACTTCTTTCC | 220 |
| chr8:15508656-15508658 | −1 < x < 0 | 3.75E−08 | ACCCTTGCCCTCCTTGGCAGGCCAGCGCTCCGGGAGGCCCTAAAGAACGGAC | 221 |
| chr8:26390136-26390138 | −1 < x < 0 | 2.00E−20 | GTGTTTACCTGGAGACTAATTCATACGGTAGTTATTGGGCTTTAAATCCAGG | 222 |
| chr8:26943273-26943275 | 0 < x < 1 | 1.51E−17 | GTGGCCAGCCTATTTAGTTCCACATCGTTCAGCCTAGATGCCCCCCACCACT | 223 |
| chr8:37559917-37559919 | 0 < x < 1 | 2.38E−18 | TCTGTAAGAGTGTTTCCCTTCGACACGGTGTATTTCTGGAAGGTTTTCTTCT | 224 |
| chr8:33509086-33509088 | 0 < x < 1 | 2.95E−94 | GGGCCAGTGAGTGCGGCTGCGCGGCCGGGAGGGCGGCGGGCGGCCCGCGGG | 225 |
| | | | C | |
| chr8:42969805-42969807 | 0 < x < 1 | 2.55E−45 | TCGCCGTCGCTGGAGCAGGCGGAGTCGGACGCGCTGGACATCAGCACCAAAG | 226 |
| chr8:52253090-52253092 | −1 < x < 0 | 1.16E−47 | GAGGTCAGCATGCGCCTCCACTCCCCGCGAGTGGACTTGGAGCGGCTCAGCA | 227 |
| chr8:67527837-67527839 | 0 < x < 1 | 9.12E−123 | GCGTGGCGCCCGAAGCGCTGCCACTCGCGCGCCAGCTTGCGCCCCTTCTGCC | 228 |
| chr8:67627712-67627714 | −1 < x < 0 | 1.67E−99 | ATTTCCTGAGTGCCCACTCTGTGCCCGACATGGGTCACACAGCTCCCCCAGG | 229 |
| chr8:70455928-70455930 | −1 < x < 0 | 6.25E−120 | GAGCCCAGGCCGCCGTGCCAGCCCACGTCACCGCAGACATACACAGACACGC | 230 |
| chr9:933885-933887 | −1 < x < 0 | 2.32E−24 | CACCTGTGCTAGTCCAGCCTGATGGCGCAGGTGCAGCCCAACACTCAGGGGG | 231 |
| chr9:1046331-1046333 | −1 < x < 0 | 4.39E−23 | AGGAAGGGGGGGCGAGGGCATGCTTCGAGTTTGGATCCGAGGTCTTCAGGGG | 232 |
| chr9:2360284-2360286 | 0 < x < 1 | 3.93E−08 | CGGGAGCCCAGCGACCCGGGGAAACCGATGTTAAGATGAAGGTATTTTCATT | 233 |
| chr9:5481047-5481049 | −1 < x < 0 | 0.413612259 | GCCTTCCCCACTCAGCGGCCGGGCCCGGCTTTCCCGCCCCACGGCCCGGCCA | 234 |
| chr9:5697426-5697428 | −1 < x < 0 | 1.47E−13 | GGCGCTGGACGGGCATTCCGGGGGGGGGGGGAGCCAGCCCGACCCCAAGCAC | 235 |
| chr9:12758035-12758037 | −1 < x < 0 | 2.63E−12 | TTGCTGATCTCTGCCCCGTGGAGCCCGCCCCCCGAGGTTCCCAGTTACACTC | 236 |
| chr9:17910388-17910390 | 0 < x < 1 | 1.44E−17 | CCGGGCTACCGGGTGCGGGGGGCAGCGGCGCCATCAGCACGGCCACCTACAC | 237 |
| chr9:12642800-12642802 | 0 < x < 1 | 1.49E−36 | GCGAGGCTGCGAGGCTGCGAGGCTGCGAGGCGCGGCGCCCCGTGGTGGTCAC | 238 |
| chr9:18277673-18277675 | 0 < x < 1 | 1.06E−41 | GCCTCGCAGCCCTGGCGCGCGCGCTCGCAGATGGAGCGGCACGGCGGGATGG | 239 |
| chr9:18277684-18277686 | 0 < x < 1 | 6.23E−49 | CTGGCGCGCGCGCTCGCAGATGGAGCGGCACGGCGGGATGGCCTGCTCCAGC | 240 |
| chr9:19703027-19703029 | −1 < x < 0 | 3.63E−24 | AAGCCCCTCCCCCAAGCCAAGGACCCGGTCACCCATCCCCTCCACCTCCCTA | 241 |
| chr9:20527917-20527919 | 0 < x < 1 | 4.14E−12 | CGAGTCCCCCTCGCACCCCGCCCCGCGGCCAGTCCCGCTCCGGAGCCCTCTG | 242 |
| chr9:25415871-25415873 | 0 < x < 1 | 5.08E−66 | GTGAGGATGCTGCTGAGCTTGCGATCGAAGGAGCCACTCATCCTGGCGAGCG | 243 |
| chr9:24331651-24331653 | −1 < x < 0 | 5.37E−15 | GATCGCCCGGCCACATGACCGGCGCCGGCCAATCGCTGGATTCAACCACTCA | 244 |
| chr9:35985983-35985985 | 0 < x < 1 | 1.12E−10 | GAGGGACCGCGAGCCGCTGATTGGTCGCAGGCCGCGGTGACGTCGGCGGGCG | 245 |
| chr9:36717168-36717170 | 0 < x < 1 | 2.42E−35 | GGCTCCGGGGCTTTTCCACCCGCCGCGAGTCCACTTTTAGTCTGTAGAGAAG | 246 |
| chr9:47196375-47196377 | 0 < x < 1 | 3.67E−33 | CCATAGCGGCTGCCCAGCTCGTCCCCGAAGCGGACCATGGTTCCCGGGCGCC | 247 |
| chr9:47612619-47612621 | −1 < x < 0 | 1.62E−34 | GGACCGGGGACCTGCAGGTCGGTCACGATGGGTGGACGTCGCTCCAGCCCTG | 248 |
| chr9:47612682-47612684 | −1 < x < 0 | 1.95E−33 | GCTCTGGGCCTGGGGACTCAGCAGACGCCTCCCCACCCGCTAGCAGGACCCT | 249 |
| chr9:47957838-47957840 | −1 < x < 0 | 0.000380125 | GGAACAGCTGGCCTGGCCTCTGCAGCGCCCAGGAGCTGGCCCGGCTGACGCC | 250 |
| chr9:48225579-48225581 | 0 < x < 1 | 4.32E−06 | CCGCCATCCCTGGGGGTCAAGTGCCCGGCCTGTCTCCTGGAGGCTCGCCACG | 251 |
| chr9:50976682-50976684 | 0 < x < 1 | 2.19E−57 | CCGGAGTGATGACGCTGATTGGCTCCGGCCGATTTTGGGGGTGATGTCACGG | 252 |
| chr9:49248845-49248847 | −1 < x < 0 | 4.75E−26 | GGTCACTGAGCTCTGGTTTCATCAGCGAGGGAACTTACTCAGCTCGAGGGAA | 253 |
| chr9:54993941-54993943 | −1 < x < 0 | 4.44E−32 | CTGCCCTGTCCCACAAGCTACCTCCCGAGTGAAATACCCATGCGCTGAGCCC | 254 |
| chr9:57421027-57421029 | 0 < x < 1 | 1.10E−47 | CGTGTTACCTTTTGCTGGGCTCCTTCGCAGTCACTTTGCAGGTCCTTCTACC | 255 |

TABLE 2
“Top3” Biological Clock

| | | p-value | | |
|---|---|---|---|---|
| Site | Clock Co-efficient | (Pr(>\|t\|) | Sequence | SEQ ID NO: |
| (Intercept) | −1.488083921 | 7.40E−19 | | |
| chr2.32494387.32494389 | 25.49884175 | 0.000801242 | TTGGAAGCTCGGGGGGCGGAGATCCCGCGCCGCCCGGCCGGCCTCGGGGCGG | 86 |
| chr6.45773846.45773848 | 18.31906431 | 2.49E−165 | GAGAAGGACGTCCCTGTCGCGAAGCCGTTCGCAGCGCGCGGCCCTGGGGGCG | 208 |
| chr16.8886545.8886547 | 15.43246339 | 1.49E−22 | GGCGGGCTCCCCGTGCGCGCACGGCCGCGGCCGACCTCTCCGCGCGGGCTCC | 63 |

TABLE 3
“Top5” Biological Clock

| | | p-value | | |
|---|---|---|---|---|
| Site | Clock Co-efficient | (Pr(>\|t\|] | Sequence | SEQ ID NO: |
| (Intercept) | −1.626008306 | 2.92E−23 | | |
| chr2.32494387.32494389 | 25.40472484 | 0.000515096 | TTGGAAGCTCGGGGGGCGGAGATCCCGCGCCGCCCGGCCGGCCTCGGGGCGG | 86 |
| chr6.45773846.45773848 | 16.83100695 | 3.51E−140 | GAGAAGGACGTCCCTGTCGCGAAGCCGTTCGCAGCGCGCGGCCCTGGGGGCG | 208 |
| chr16.8886545.8886547 | 14.1755269 | 2.09E−20 | GGCGGGCTCCCCGTGCGCGCACGGCCGCGGCCGACCTCTCCGCGCGGGCTCC | 63 |
| chr5.61645225.61645227 | 9.685390237 | 1.46E−06 | TAAAGGGGCGATGCCGCGACCCAGGCGCTGGCGTGGGGTCCACCGGCGTCGC | 196 |
| chr15.10856498.10856500 | 20.454796 | 4.36E−10 | CCCCAGCCGGGCCGGCTCGGGTTTCCGACGGAGGGCCGGGCTGCGCGGCGTC | 57 |

TABLE 4
“Top10” Biological Clock

| | | p-value | | |
|---|---|---|---|---|
| Site | Clock Co-efficient | (Pr(>\|t\|) | Sequence | SEQ ID NO: |
| (Intercept) | 2.636785028 | 1.68E−11 | | |
| chr2.32494387.32494389 | 31.48707725 | 3.15E−06 | TTGGAAGCTCGGGGGGCGGAGATCCCGCGCCGCCCGGCCGGCCTCGGGGCGG | 86 |
| chr6.45773846.45773848 | 14.11743561 | 1.46E−118 | GAGAAGGACGTCCCTGTCGCGAAGCCGTTCGCAGCGCGCGGCCCTGGGGGCG | 208 |
| chr16.8886545.8886547 | 11.15446753 | 3.81E−16 | GGCGGGCTCCCCGTGCGCGCACGGCCGCGGCCGACCTCTCCGCGCGGGCTCC | 63 |
| chr5.61645225.61645227 | 7.635417929 | 1.71E−05 | TAAAGGGGCGATGCCGCGACCCAGGCGCTGGCGTGGGGTCCACCGGCGTCGC | 196 |
| chr15.10856498.10856500 | 15.04348128 | 2.25E−07 | CCCCAGCCGGGCCGGCTCGGGTTTCCGACGGAGGGCCGGGCTGCGCGGCGTC | 57 |
| chr33.26512711.26512713 | 9.624991584 | 2.86E−05 | CGTCGCCGCCGCGCTCCTGCCAGGCCGACCTGGAGCGGAAGCTGAAGCCGAA | 162 |
| chr20.57347921.57347923 | −8.924503605 | 7.22E−31 | ACCAGCCGCCCAGCCGTGACCTTTCCGGGCCTGTTCCTGCGGCTCTGGGCGC | 106 |
| chr12.12684190.12684192 | 10.01399267 | 0.004659424 | CTCAGCCGCTTGGGCGGGTATCCACCGGCCCCGGGGGTAGTCGGCCCCTGCG | 37 |
| chr24.30860619.30860621 | −6.97439064 | 0.178939513 | CAGAACCGCGCTTCCCCGAAACTCGCGGGCGGTGGGATCGCAAGTCACCCCC | 120 |
| chr33.26512692.26512694 | 5.841178421 | 3.34E−06 | CGGCTGCCGCGCCCGCAGTCGTCGCCGCCGCGCTCCTGCCAGGCCGACCTGG | 161 |

TABLE 5
“Top20” Biological Clock

| Site | Clock Co-efficient | p-value (Pr(>\|t\|) | Sequence | SEQ ID NO: |
|---|---|---|---|---|
| (Intercept) | 1.426173487 | 0.000738149 | | |
| chr2.32494387.32494389 | 29.97454506 | 1.89E−07 | TTGGAAGCTCGGGGGGCGGAGATCCCGCGCCGCCCGGCCGGCCTCGGGGCGG | 86 |
| chr6.45773846.45773848 | 8.47941593 | 1.94E−47 | GAGAAGGACGTCCCTGTCGCGAAGCCGTTCGCAGCGCGCGGCCCTGGGGGCG | 208 |
| chr16.8886545.8886547 | 5.880759211 | 1.03E−06 | GGCGGGCTCCCCGTGCGCGCACGGCCGCGGCCGACCTCTCCGCGCGGGCTCC | 63 |
| chr5.61645225.61645227 | 4.434791641 | 0.003872156 | TAAAGGGGCGATGCCGCGACCCAGGCGCTGGCGTGGGGTCCACCGGCGTCGC | 196 |
| chr15.10856498.10856500 | 10.82275414 | 1.28E−05 | CCCCAGCCGGGCCGGCTCGGGTTTCCGACGGAGGGCCGGGCTGCGCGGCGTC | 57 |
| chr33.26512711.26512713 | 6.576079765 | 0.000762612 | CGTCGCCGCCGCGCTCCTGCCAGGCCGACCTGGAGCGGAAGCTGAAGCCGAA | 162 |
| chr20.57347921.57347923 | −7.357261642 | 9.37E−28 | ACCAGCCGCCCAGCCGTGACCTTTCCGGGCCTGTTCCTGCGGCTCTGGGCGC | 106 |
| chr12.12684190.12684192 | 6.041563837 | 0.045272957 | CTCAGCCGCTTGGGCGGGTATCCACCGGCCCCGGGGGTAGTCGGCCCCTGCG | 37 |
| chr24.30860619.30860621 | −4.447714718 | 0.314850718 | CAGAACCGCGCTTCCCCGAAACTCGCGGGCGGTGGGATCGCAAGTCACCCCC | 120 |
| chr33.26512692.26512694 | 2.226277287 | 0.041402225 | CGGCTGCCGCGCCCGCAGTCGTCGCCGCCGCGCTCCTGCCAGGCCGACCTGG | 161 |
| chr10.7411293.7411295 | 3.014021236 | 2.67E−08 | CAGCACCGGGCATCTGGACAGCAACCGGAGAGCGAGGCGTGTTTCTCCTGGT | 15 |
| chr1.22213697.22213699 | 4.119609069 | 0.001713821 | TGACCCCGAGGGAGCCGCACCCCGGCGCCGCGACGTTCACCTCCGGGGGCGC | 2 |
| chr5.32711703.32711705 | 10.34618246 | 0.000750409 | GGTATCCCGTCCCCTCGGCTCGGCTCGGCTCGGCTCGGCTCGGCGGGCCCCG | 189 |
| chr24.21051024.21051026 | 1.754900433 | 0.014548112 | ACGTTGCTCTTCGTGCTCCTGCCCTCGCTGGTCGTGCAGCTGCTCAGCTTCC | 119 |
| chr5.21480151.21480153 | 5.475715773 | 0.001332943 | TTGAGCTGCCTGGAGCAGCAGCCGTCGGCCACCTAGCAGGAGTGTCCTCGCA | 186 |
| chr17.33851462.33851464 | 5.28692709 | 3.41E−07 | ACAGCAGGATGAAGCCGAAAGCCTCCGAGTGCCGGTCGAAGAAGTACTCGTT | 68 |
| chr3.46843750.46843752 | 4.070393226 | 1.33E−10 | CCAGGGCCACAGACCTGGAAGTCGCCGGGAGCCCGGCAGCACTCCTCTCAAG | 147 |
| chr13.37474592.37474594 | −4.646099678 | 2.64E−11 | TTCCCGGCTCCCGAACCAGGGTGGCCGAGCCAGGCCTCTGCCTCCGGGGGTC | 44 |
| chr33.19810904.19810906 | 2.651938013 | 0.001641489 | GCGCTCCTCGCCGCTCTCGGTGGCCCGGAGAACAAAAGCCCCGGGCCTGGCC | 163 |
| chr20.44942213.44942215 | 4.772633102 | 7.94E−06 | GGCAGGGCCGCCAGGCGCCGGCCCCCGGTGAAGAAGTCCTCGGCGCTGCGCT | 98 |

1. A method for selecting a lifestyle regime, dietary regime or therapeutic intervention for a dog, the method comprising:
a) providing a DNA methylation profile from a sample obtained from the dog;
b) determining a biological age of the dog using the DNA methylation profile; and
c) selecting a suitable lifestyle regime, dietary regime or therapeutic intervention for the dog based on the biological age determined in step b).

2. A method for determining the efficacy of a lifestyle regime, dietary regime or therapeutic intervention for improving the biological age of a dog, said method comprising:
a) applying a lifestyle regime, dietary regime or therapeutic intervention to the dog, wherein the lifestyle regime, dietary regime or therapeutic intervention has been selecting according to the method of claim 1;
b) after a time period of applying the lifestyle regime, dietary regime or therapeutic intervention to the dog; determining a biological age of the dog using a DNA methylation profile from a sample obtained from the dog;
c) determining if there has been a change in the biological age of the dog after the time period of following the lifestyle regime, dietary regime or therapeutic intervention.

3-5. (canceled)

6. A method for preventing or reducing the risk of a dog developing a disease; the method comprising:
a) determining a biological age of the dog using a DNA methylation profile from a sample obtained from the dog; wherein the biological age determined for the dog is associated with an increased likelihood to develop the disease; and
b) selecting a lifestyle regime, dietary regime or therapeutic intervention for the dog based on the biological age determined in step a);
wherein the lifestyle regime, dietary regime or therapeutic intervention prevents or reduces the risk of the dog developing the disease; preferably wherein the disease is an age-related disease.

7-8. (canceled)

9. A method according to claim 1 wherein the lifestyle regime, dietary regime or therapeutic intervention is selected based on a determination that the dog has an increased biological age compared to its chronological age.

10. A method according to claim 1 wherein the lifestyle regime, dietary regime or therapeutic intervention is a dietary intervention.

11. A method according to claim 10 wherein the dietary intervention is a calorie-restricted diet, a senior diet or a low protein diet.

12. The method according to claim 1 wherein the sample is a blood sample.

13. The method according to claim 1, wherein step a) further comprises determining a DNA methylation profile from a sample obtained from the dog.

14. The method according to claim 1, wherein the DNA methylation profile comprises at least one methylation site as listed in Table 1.

15. The method according to claim 14, wherein the DNA methylation profile comprises at least 3, each of the methylation sites as listed in Table 1.

16-19. (canceled)

20. The method according to claim 13 wherein DNA methylation is determined using a method which comprises one or more of the following steps: (a) treating the sample DNA with APOBEC to deaminate cytosines; (b) a capture-based enrichment; and/or (c) high throughput sequencing.

21. A method according to claim 7 wherein the DNA methylation profile is associated with increased biological age of (i) a tissue; (ii) an organ; or (iii) a physiological system, such as the immune, gastrointestinal, urinary, muscular, cardiovascular, and/or neurological system.

22. A method according to claim 21, further comprising applying to the dog a lifestyle regime, dietary regime or therapeutic intervention which is suitable for improving the biological age of the tissue; organ; or physiological system for which an increased biological age has been determined.

23-36. (canceled)
